(19) United States
(12) Patent Application Publication
(10) Pub. No.: US 2004/0152759 A1
(43) Pub. Date: Aug. 5, 2004

(54) Combination administration of an indolinone with a chemotherapeutic agent for cell proliferation disorders

(75) Inventors: Tinya Abrams, Richmond, CA (US); Lesley Murray, San Jose, CA (US); Nancy Pryer, Kensington, CA (US); Julie Cherrington, San Francisco, CA (US)

Correspondence Address: FOLEY AND LARDNER, SUITE 500, 3000 K STREET NW, WASHINGTON, DC, 20007, US

(73) Assignee: SUGEN, Inc.

(21) Appl. No.: 10/712,296

(22) Filed: Nov. 14, 2003

Related U.S. Application Data
(60) Provisional application No. 60/426,386, filed on Nov. 15, 2002.

(51) Int. Cl. A61K 31/4439; A61K 31/405

(52) U.S. Cl. 514/414; 514/339

(57) ABSTRACT

The invention relates to a method of treating cancer by administering a combination of an indolinone compound with another chemotherapeutic agent. The combination of an indolinone compound of Formula I:

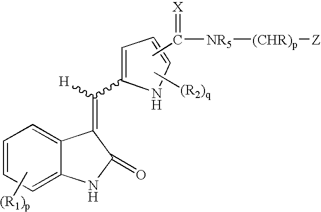

with another chemotherapeutic agent provides an enhanced effect in treating cancer patients.

### RELATED APPLICATIONS

This application claims priority to provisional applications 60/426,386 filed Nov. 15, 2002, the entire content of which is incorporated herein by reference.

### FIELD OF THE INVENTION

The invention relates to a method of treating cell proliferation disorders such as cancer by administering a combination of an indolinone compound with another chemotherapeutic agent. The combination of an indolinone compound of Formula I with another chemotherapeutic agent provides an enhanced effect in treating certain types of cancer patients.

### BACKGROUND OF THE INVENTION

Breast cancer is a type of cancer where cells in the breast tissue divide and grow without control. About 80 percent of cases of breast cancer originate in the mammary ducts, while about 20 percent arise in the lobules. Invasive breast cancer occurs when abnormal cells from inside the lobules or ducts break out into the surrounding tissue. This term, though, does not necessarily mean that metastases have been found anywhere beyond the breast. When invasive cancer is generally at its most treatable, such as when a tumor is relatively small and has not spread to the lymph nodes, it is considered “early stage.” When the condition is more serious and successful treatment less likely, such as when a tumor is very large or has spread to other organs (like the liver, lungs, and bones), it is considered “advanced stage”.

When abnormal cells grow inside the lobules or milk ducts but there is no sign that the cells have spread out to the surrounding tissue or beyond, the condition is called carcinoma in situ. There are two main categories of carcinoma in situ: ductal carcinoma in situ and lobular carcinoma in situ.

Normally the mammary ducts are hollow so that fluid can pass through them. With Ductal Carcinoma In Situ (DCIS), excess cells that are very similar to invasive cancer cells grow inside the ducts. DCIS is not invasive cancer, but it is associated with an increased risk of breast cancer and is considered a precancerous condition that has the potential to eventually develop into invasive cancer.

Like the milk ducts, the lobules of the breast tissue have open space inside them. When large numbers of abnormal cells grow in the lobules, the condition is called Lobular Carcinoma In Situ (LCIS). LCIS is not invasive cancer, and it is not a direct cancer precursor, that is, the abnormal cells found inside the lobules are not likely to turn into cancer later on. LCIS is, however, a risk factor for invasive cancer.

Most women with stage 1 or 2 breast cancer are treated with a combination of surgery, radiation therapy, and/or adjuvant systemic therapy, which is treatment given in addition to surgery and radiation to eliminate tumors that may have spread to other sites. There are two types, chemotherapy and hormone therapy.

More than 30 different drugs are commonly used for chemotherapy. The most effective of these drugs, known as first-line drugs, are doxorubicin, epirubicin, methotrexate, cyclophosphamide, 5-fluorouracil, docetaxel and paclitaxel. Although each of these individual drugs has shown some efficacy on its own, Applicants' research has shown that combining different drugs further increases their ability to kill cancer cells. Some of the currently available combinations of chemotherapy for adjuvant therapy are:

1. a combination of cyclophosphamide and doxorubicin (Adriamycin).

2. a combination of cyclophosphamide, methotrexate and 5-fluorouracil.

3. CAF (FAC), a combination of cyclophosphamide, doxorubicin (Adriamycin) 5-fluorouracil.

4. a combination of cyclophosphamide, doxorubicin (Adriamycin) and paclitaxel (Taxol).

5. a combination of cyclophosphamide, doxorubicin (Adriamycin) and taxotere (Docetaxel).

Colon cancer involves a growth of abnormal or malignant cells within the lining of the colon or rectum. The majority of colon cancers arise from non-malignant growths known as adenomas. In some cases, adenomas have the potential to increase in size and undergo a series of changes within the cells, resulting in them becoming abnormal in function, structure and shape. This is commonly referred to as a malignancy or a cancer. Current treatment regimens for colon cancer involve surgery to remove the tumor, radiation and chemotherapy. Chemotherpapy given for colon cancer usually consists of variations on two drug regimens, fluorouracil (5-FU) and levamisole, and 5-FU and leucovorin.

Small cell lung carcinoma (SCLC) is distinctive from other kinds of lung cancer (metastases are already present at the time of discovery) and accounts for approximately 110,000 cancer diagnoses annually. A deletion of part of chromosome 3 was first observed in 1982 in small cell lung carcinoma cell lines. As with other cancers, mutations in a variety of molecules (oncogenes and tumor-suppressor genes) that control cell growth and division are observed, and no one mutation is likely to result in cancerous growth. Three kinds of treatment are conventionally used: surgery, radiation therapy and chemotherapy. While no single chemotherapy regimen is considered standard, those that have shown activity include oral etoposide, etoposide/cisplatin, cyclophosphamide/doxorubicin/vincristine (CAV), lomustine/methotrexate, and topotecan and combinations thereof.

Non small cell lung cancer (NSCLC) is a group of lung cancers that includes squamous cell carcinoma, also called epidermoid carcinoma, adenocarcinoma, adenosquamous carcinoma, large cell carcinoma, and undifferentiated carcinoma.

Renal cell cancer (also called cancer of the kidney or renal adenocarcinoma) is a disease in which cancer (malignant) cells are found in certain tissues of the kidney.

Gastrointestinal stromal tumors (GIST) are a type of tumor that usually begins in cells in the wall of the gastrointestinal tract. It can be benign or malignant.

Thyroid cancer involves malignant tumors of the thyroid.

Sarcomas includes any cancers of the bone, cartilage, fat, muscle, blood vessels, or other connective or supportive tissue.

Neuroendocrine tumors refer to the type of cell that a tumor grows from rather than where that tumor is located. Neuroendocrine cells produce hormones or regulatory proteins, and so tumors of these cells usually have symptoms that are related to the specific hormones that they produce.

Overall, there are many cases where known chemotherapeutic agents fail to eradicate cancer due to acquired resistance of the cancer to the agent. Applicants have determined that compounds of Formula I in combination with another chemotherapeutic agent may be administered at a dose lower than the current standard while still providing beneficial efficacy and perhaps reducing toxicity of the chemotherapeutic agent to the patient.

### SUMMARY OF THE INVENTION

One embodiment of the present invention relates to a method of treating cancer comprising administering to a patient in need thereof an effective amount of a compound of Formula I:

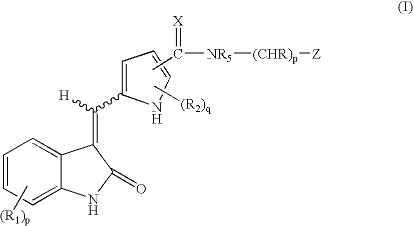

wherein,

each R is independently hydrogen, hydroxy, alkyl, aryl, cycloalkyl, heteroaryl, alkoxy, heterocyclic or amino;

each R₁ is independently alkyl, halo, alkoxy, haloalkyl, haloalkoxy, cycloalkyl, heterocyclic, hydroxy, —C(O)—R₈, —NR₉R₁₀, —NR₉C(O)—R₁₂ or —C(O)NR₉R₁₀;

each R₂ is independently alkyl, aryl, heteroaryl, —C(O)—R₈ or SO₂R″, where R″ is alkyl, aryl, heteroaryl, NR₉N₁₀ or alkoxy;

each R₅ is independently hydrogen, alkyl, aryl, haloalkyl, cycloalkyl, heteroaryl, heterocyclic, hydroxy, —C(O)—R₈ or (CHR)ᵣR₁₁;

X is O or S;

j is 0 or 1;

p is 0, 1, 2 or 3;

q is 0, 1 or 2;

r is 0, 1, 2or 3;

R₈ is hydroxy, alkyl, aryl, heteroaryl, alkoxy, cycloalkyl or heterocyclic;

R₉ and R₁₀ are independently hydrogen, alkyl, aryl, aminoalkyl, heteroaryl, cycloalkyl and heterocyclic, or R₉ and R₁₀ together with N may form a ring, where the ring atoms are selected from the group consisting of C, N, O and S;

R₁₁ is hydroxy, amino, monosubstituted amino, disubstituted amino, alkyl, aryl, heteroaryl, alkoxy, cycloalkyl or heterocyclic

R₁₂ is alkyl, aryl, heteroaryl, alkoxy, cycloalkyl or heterocyclic; and

Z is hydroxy, —O-alkyl, or —NR₃R₄, where R₃ and R₄ are independently hydrogen, alkyl, aryl, heteroaryl, cycloalkyl, or heterocyclic, or R₃ and R₄ may combine with N to form a ring where the ring atoms are selected from the group consisting of CH₂, N, O and S, or

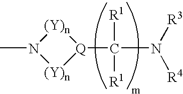

wherein Y is independently CH₂, O, N or S, Q is C or N, n is independently 0, 1, 2, 3 or 4, and m is 0, 1, 2or 3;

or a pharmaceutically acceptable salt, hydrate or solvate thereof, in combination with at least one chemotherapeutic agent selected from the group consisting of microtubule interference agents, topoisomerase inhibitors, alkylating agents, thymidylate synthase inhibitors, irreversible steroidal aromatase inactivators, anti-metabolites, pyrimidine antagonists, purine antagonists, ribonucleotide reductase inhibitors, and kinase inhibitors. Compounds of Formula I and their preparation are described in WO 02/066463 and U.S. Pat. No. 6,573,293, the disclosures of which are incorporated herein by reference in their entireties.

In another embodiment, R₁ is halo, preferably F or Cl, and p is 1.

In another embodiment, Z is —NR₃R₄, wherein R₃ and R₄ are lower alkyl or form a morpholine ring.

In another embodiment, Z is:

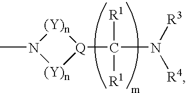

wherein each Y is CH₂, each n is 2, m is 0 and R₃ and R₄ form a morpholine ring.

In another embodiment, R₂ is methyl and q is 2, wherein the methyls are bonded at the 3 and 5 positions.

In another embodiment, the compound of formula I is selected from the group consisting of

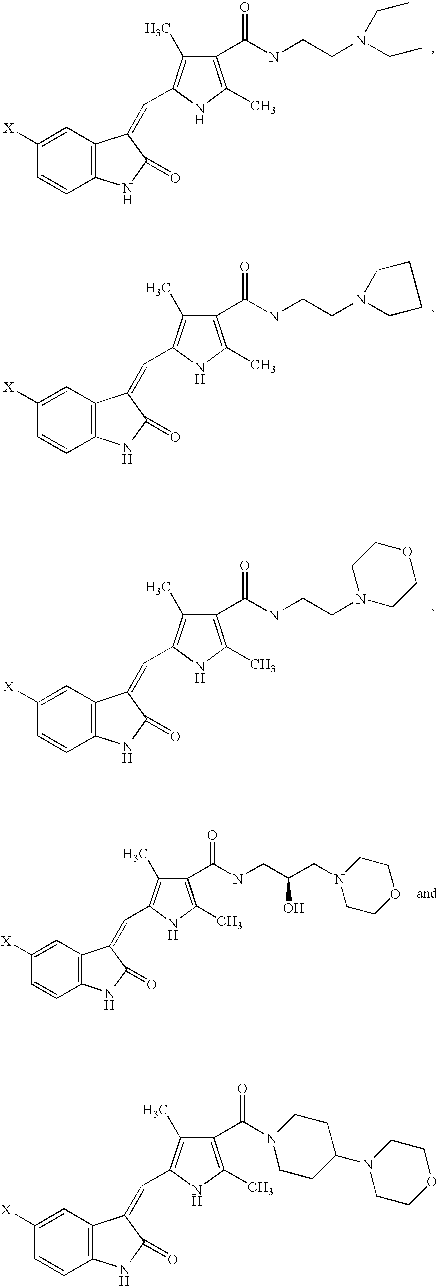

and pharmaceutically acceptable salts, solvates and hydrates thereof.

In another embodiment, the compound of formula I is selected from the group consisting of:

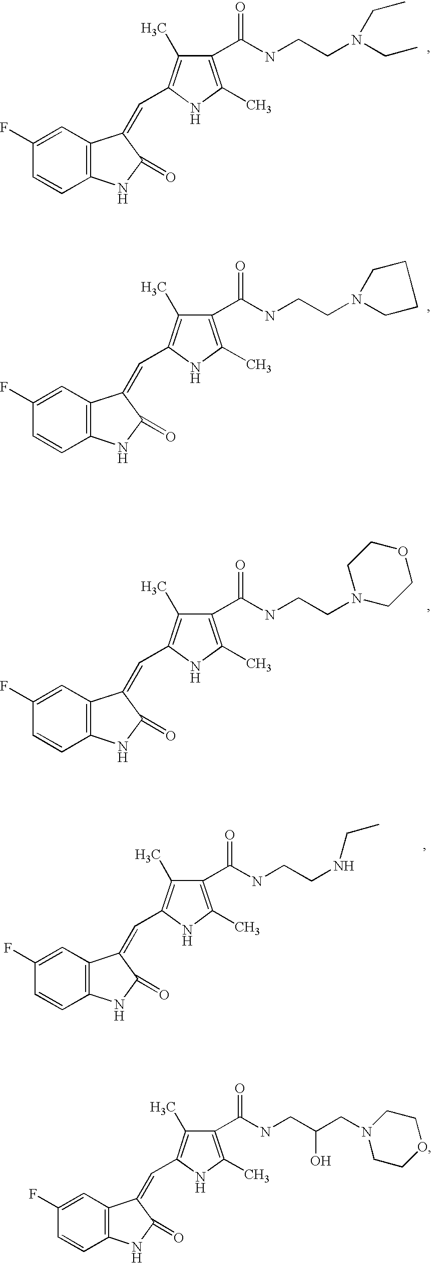

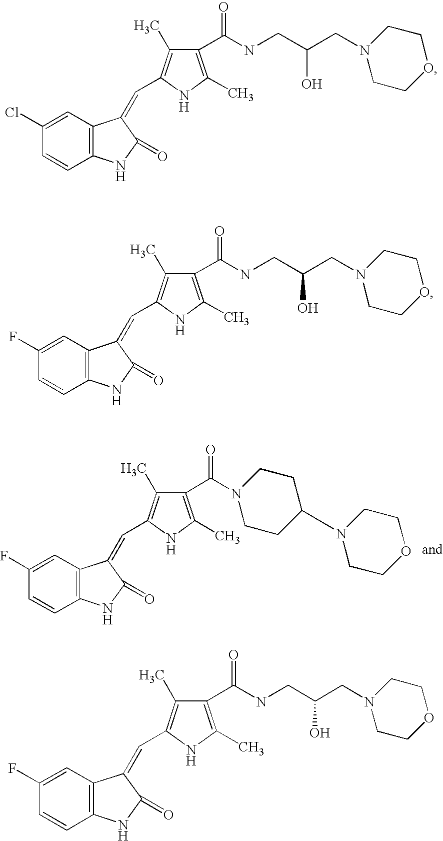

and pharmaceutically acceptable salts, hydrates and solvates thereof

In another embodiment, the compound is

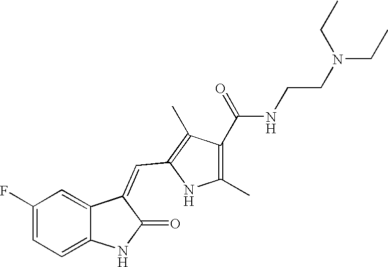

or a pharmaceutically acceptable salt, hydrate or solvate thereof.

In a preferred aspect of this embodiment, the salt is a malate salt, preferably an L-malate salt.

In a preferred aspect of any of the preceding embodiments, the at least one chemotherapeutic agent is selected from the group consisting of paclitaxel, docetaxel, vinblastine, vincristine, vindesine, irinotecan, doxorubicin, epirubicin, leucovorin, etopside, teniposide, idarubicine, gemcitabine, daunorubicin, carboplatin, cisplatin, oxaliplatin, chlorambucil, melphalan, cyclophosphamide, ifosfamide, temozolomide, thiotepa, mitomycin C, busulfan, carmustine, lomustine, 5-fluorouracil, capecitabine, AROMASIN™ (exemestane), methotrexate, trimetrexate, fluorouracil, fluorodeoxyuridine, azacytidine, mercaptopurine, thioguanine, pentostatin, cytarabine, fludarabine, hydroxyurea, AVASTIN™ (bevacizumab) cetuximab, IRESSA™ (gefitinib) and GLEEVEC™ (imatinib).

In another embodiment, at least two additional chemotherapeutic agents are used in combination with a compound of Formula I.

In another embodiment, at least three additional chemotherapeutic agents are used in combination with a compound of Formula I.

In an embodiment of the invention, an additional agent can be administered in the methods of the invention with the compound of Formula I. This additional agent is not in itself a chemotherapeutic agent but has a therapeutic effect, such as, for example, a nutraceutical which can improve side effects (like cachexia) from conventional chemotherapy.

### BRIEF DESCRIPTION OF THE DRAWINGS

FIG. 1 is a graph showing combination of Compound 1 and docetaxel administered at 5 mg/kg/day with results of tumor growth delay compared to monotherapies.

FIG. 2 is a graph showing combination of Compound 1 and docetaxel administered at 10 mg/kg/day with results of tumor growth delay compared to monotherapies.

FIG. 3 is a graph showing combination of Compound 1 and docetaxel administered at 15 mg/kg/day with results of tumor growth delay compared to monotherapies.

FIG. 4 is a graph showing combination of Compound 1 and docetaxel administered at 5, 10 and 15 mg/kg/day with results of tumor growth delay compared to monotherapies.

FIG. 5 is a graph showing combination of Compound 1 and 5-FU with results of tumor growth delay compared to monotherapies.

FIG. 6 is a graph showing combination of Compound 1 and Doxorubicin with results of tumor growth delay compared to monotherapies.

FIG. 7 is a graph showing combination of Compound 1 and Cisplatin with results of tumor growth delay compared to monotherapies.

FIG. 8 is a graph showing combination of Compound 1 and CPT-11, administered at 20 mg/kg/day and 40 mg/kg/day, with results of tumor growth delay compared to monotherapies.

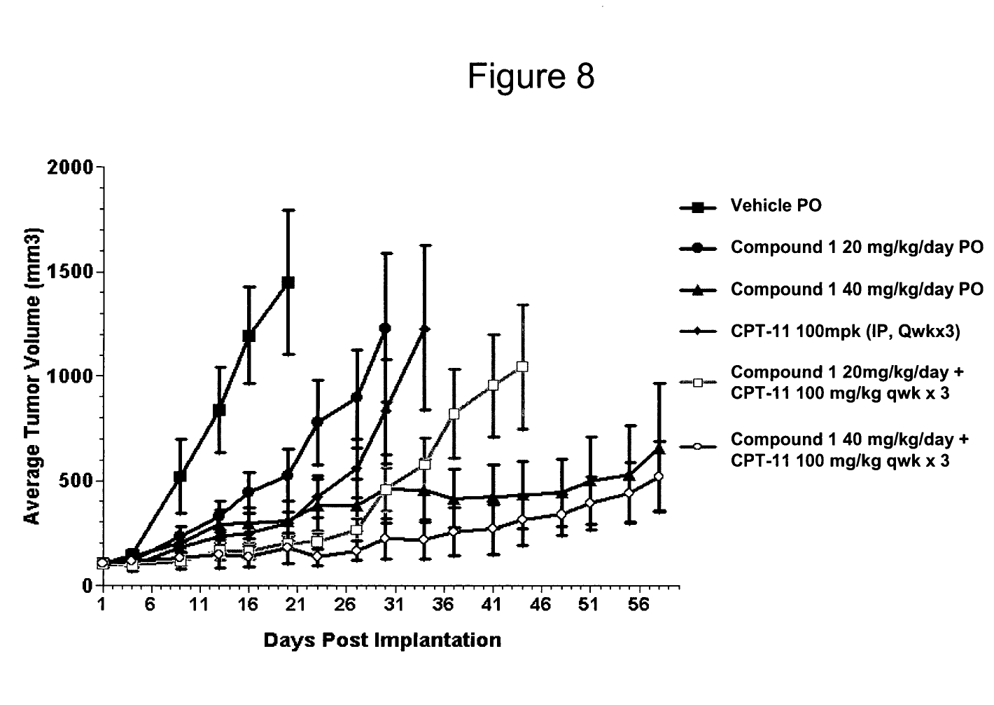

### DETAILED DESCRIPTION OF THE PREFERRED EMBODIMENTS

The compounds of formula I are useful in the treatment of patients with cancer. In particular, they are useful in the treatment of cancer patients with because of the activity of the present compounds of formula I as receptor tyrosine kinase (RTK) inhibitors. In particular, the compounds of formula I are inhibitors of KIT and FLT3 and the receptors for VEGF and PDGF. The compounds of formula I block both RTKs expressed directly in tumor cells and those RTKs expressed in endothelial or stromal cells which leads to their ability to inhibit tumor growth.

Chemotherapeutic agents contemplated for administration with the indolinone compounds of Formula I include but are not limited to microtubule interference agents, topoisomerase inhibitors, alkylating agents, thymidylate synthase inhibitors, irreversible steroidal aromatase inactivators, anti-metabolites, pyrimidine antagonists, purine antagonists, ribonucleotide reductase inhibitors, and kinase inhibitors.

Microtubule interference agents are those agents which induce disorganized microtubule formation, disrupting mitosis and DNA synthesis and include the taxanes, for example, paclitaxel and docetaxel; vinca alkyloids such as vinblastine, vincristine and vindesine.

Topoisomerase inhibitors which act by breaking DNA, include two types, topoisomerase I and topoisomerase II inhibitors. Topoisomerase I inhibitors include but are not limited to irinotecan (CPT-11). Topoisomerase II inhibitors include, e.g., doxorubicin and epirubicin. Other toposiomerase inhibitors useful in the present invention include but are not limited to etopside, teniposide, idarubicin and daunorubicin.

Alkylating agents which act by damaging DNA, such as chlorambucil, melphalan, cyclophosphamide, ifosfamide, temozolomide, thiotepa, mitomycin C, busulfan, carmustine (BCNU) and lomustine (CCNU) have been shown to be useful chemotherapy agents. The alkylating agents also include the platins such as carboplatin and cisplatin which have been shown to be useful chemotherapy agents, even though they are not alkylators, but rather act by covalently bonding DNA.

Thymidylate synthase inhibitors, which interfere with transcription by metabolizing to false bases of DNA and RNA, include, e.g., 5-fluorouracil and capecitabine.

Irreversible steroidal aromatase inhibitors, which act as false substrates for the aromatase enzyme, include but are not limited to AROMASIN®.

Anti-metabolites such as folate antagonists, methotrexate and trimetrexate (Alimta) have been found to be useful as chemotherapeutic agents.

Pyrimidine antagonists such as fluorouracil, fluorodeoxyuridine and azacytidine have been found to be useful as chemotherapeutic agents.

Purine antagonists have been found to be useful as chemotherapeutic agents and include agents such as mercaptopurine, thioguanine and pentostatin. Sugar modified analogs also useful as chemotherapeutic agents include cytarabine and fludarabine.

Ribonucleotide reductase inhibitors have been found to be useful as chemotherapeutic agents and include agents such as hydroxyurea.

In addition to the above recited conventional chemotherapeutic agents, the compounds of Formula I can be used in combination with other kinase inhibitors, such as AVASTIN™ (bevacizumab), cetuximab, IRESSA™ (gefitinib) and GLEEVEC™ (imatinib).

In a preferred embodiment of the invention, the additional chemotherapeutic agent administered in combination with the compound of formula I is a taxane, more preferably paclitaxel or docetaxel.

In a preferred embodiment of the invention, the additional chemotherapeutic agent administered in combination with the compound of formula I is a topoisomerase inhibitor, more preferably a topoisomerase I or topoisomerase II inhibitor, more preferably an anthracycline and more preferably doxorubicin or epirubicin and combinations thereof.

In a preferred embodiment of the invention, the additional chemotherapeutic agent administered in combination with the compound of formula I is a thymidylate synthase inhibitore, more preferably 5-fluorouracil (5-FU) or capecitabine, more preferably 5-FU.

In a preferred embodiment of the invention, the additional chemotherapeutic agent administered in combination with the compound of formula I for small cell lung cancer is an alkylating agent, more preferably cisplatin.

In a preferred embodiment of the invention, the additional chemotherapeutic agent administered in combination with the compound of formula I is an irreversible steroidal aromatse inactivator, such as AROMASIN™ (exemestane).

In a preferred embodiment, the compound of Formula I administered to a patient in need of such combination therapy is selected from the group consisting of:

5-(5-Fluoro-2-oxo-1,2-dihydro-indol-3-ylidenemethyl)-2,4-dimethyl-1H-pyrrole-3-carboxylic acid(2-diethylamino-ethyl)-amide;

5-(5-Fluoro-2-oxo-1,2-dihydro-indol-3-ylidenemethyl)-2,4-dimethyl-1H-pyrrole-3-carboxylic acid(2-pyrrolidin-1-yl-ethyl)-amide;

5-(5-Fluoro-2-oxo-1,2-dihydro-indol-3-ylidenemethyl)-2,4-dimethyl-1H-pyrrole-3-carboxylic acid(2-morpholin-4-yl-ethyl)-amide;

(S)-5-(5-Fluoro-2-oxo-1,2-dihydro-indol-3-ylidenemethyl)-2,4-dimethyl-1H-pyrrole-3-carboxylic acid(2-hydroxy-3-morpholin-4-yl-propyl)-amide;

(R)-5-(5-Fluoro-2-oxo-1,2-dihydro-indol-3-ylidenemethyl)-2,4-dimethyl-1H-pyrrole-3-carboxylic acid(2-hydroxy-3-morpholin-4-yl-propyl)-amide;

5-(5-Fluoro-2-oxo-1,2-dihydro-indol-3-ylidenemethyl)-2,4-dimethyl-1H-pyrrole-3-carboxylic acid(2-hydroxy-3-morpholin-4-yl-propyl)-amide;

5-(5-Chloro-2-oxo-1,2-dihydro-indol-3-ylidenemethyl)-2,4-dimethyl-1H-pyrrole-3-carboxylic acid(2-hydroxy-3-morpholin-4-yl-propyl)-amide;

5-(5-Fluoro-2-oxo-1,2-dihydro-indol-3-ylidenemethyl)-2,4-dimethyl-1H-pyrrole-3-carboxylic acid(2-ethylamino-ethyl)-amide and

3-[3,5-dimethyl-4-(4-morpholin-4-yl-piperidine-1-carbonyl)-1H-pyrrol-2-methylene]-5-fluoro-1,3-dihydro-indol-2-one.

In order to clearly set forth the compounds of Formula I, useful in the inventive method, the following definitions are provided.

“Alkyl” refers to a saturated aliphatic hydrocarbon radical including straight chain and branched chain groups of 1 to 20 carbon atoms (whenever a numerical range; e.g. “1-20”, is stated herein, it means that the group, in this case the alkyl group, may contain 1 carbon atom, 2 carbon atoms, 3 carbon atoms, etc. up to and including 20 carbon atoms). Alkyl groups containing from 1 to 4 carbon atoms are referred to as lower alkyl groups. When said lower alkyl groups lack substituents, they are referred to as unsubstituted lower alkyl groups. More preferably, an alkyl group is a medium size alkyl having 1 to 10 carbon atoms e.g., methyl, ethyl, propyl, 2-propyl, n-butyl, iso-butyl, tert-butyl, pentyl, and the like. Most preferably, it is a lower alkyl having 1 to 4 carbon atoms e.g., methyl, ethyl, propyl, 2-propyl, n-butyl, iso-butyl, or tert-butyl, and the like. The alkyl group may be substituted or unsubstituted. When substituted, the substituent group(s) is preferably one or more, more preferably one to three, even more preferably one or two substituent(s) independently selected from the group consisting of halo, hydroxy, unsubstituted lower alkoxy, aryl optionally substituted with one or more groups, preferably one, two or three groups which are independently of each other halo, hydroxy, unsubstituted lower alkyl or unsubstituted lower alkoxy groups, aryloxy optionally substituted with one or more groups, preferably one, two or three groups which are independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more groups, preferably one, two or three groups which are independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and the nitrogen atoms in the group being optionally substituted with one or more groups, preferably one, two or three groups which are independently of each other halo, hydroxy, unsubstituted lower alkyl or unsubstituted lower alkoxy groups, 5- or 6-member heterocyclic group having from 1 to 3 heteroatoms selected from the group consisting of nitrogen, oxygen and sulfur, the carbon and nitrogen (if present) atoms in the group being optionally substituted with one or more groups, preferably one, two or three groups which are independently of each other halo, hydroxy, unsubstituted lower alkyl or unsubstituted lower alkoxy groups, mercapto, (unsubstituted lower alkyl)thio, arylthio optionally substituted with one or more groups, preferably one, two or three groups which are independently of each other halo, hydroxy, unsubstituted lower alkyl or alkoxy groups, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)O—, and —NR₁₃R₁₄, wherein R₁₃ and R₁₄ are independently selected from the group consisting of hydrogen, unsubstituted lower alkyl, trihalomethyl, cycloalkyl, heterocyclic and aryl optionally substituted with one or more, groups, preferably one, two or three groups which are independently of each other halo, hydroxy, unsubstituted lower alkyl or unsubstituted lower alkoxy groups.

Preferably, the alkyl group is substituted with one or two substituents independently selected from the group consisting of hydroxy, 5- or 6-member heterocyclic group having from 1 to 3 heteroatoms selected from the group consisting of nitrogen, oxygen and sulfur, the carbon and nitrogen (if present) atoms in the group being optionally substituted with one or more groups, preferably one, two or three groups which are independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and the nitrogen atoms in the group being optionally substituted with one or more groups, preferably one, two or three groups which are independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more groups, preferably one, two or three groups which are independently of each other halo, hydroxy, unsubstituted lower alkyl or unsubstituted lower alkoxy groups, or —NR₁₃R₁₄, wherein R₁₃ and R₁₄ are independently selected from the group consisting of hydrogen and alkyl. Even more preferably the alkyl group is substituted with one or two substituents which are independently of each other hydroxy, dimethylamino, ethylamino, diethylamino, dipropylamino, pyrrolidino, piperidino, morpholino, piperazino, 4-lower alkylpiperazino, phenyl, imidazolyl, pyridinyl, pyridazinyl, pyrimidinyl, oxazolyl, triazinyl, and the like.

“Cycloalkyl” refers to a 3 to 8 member all-carbon monocyclic ring, an all-carbon 5-member/6-member or 6-member/6-member fused bicyclic ring or a multicyclic fused ring (a “fused” ring system means that each ring in the system shares an adjacent pair of carbon atoms with each other ring in the system) group wherein one or more of the rings may contain one or more double bonds but none of the rings has a completely conjugated pi-electron system.

Examples, without limitation, of cycloalkyl groups are cyclopropane, cyclobutane, cyclopentane, cyclopentene, cyclohexane, cyclohexadiene, adamantane, cycloheptane, cycloheptatriene, and the like. A cycloalkyl group may be substituted or unsubstituted. When substituted, the substituent group(s) is preferably one or more, more preferably one or two substituents, independently selected from the group consisting of unsubstituted lower alkyl, trihaloalkyl, halo, hydroxy, unsubstituted lower alkoxy, aryl optionally substituted with one or more, preferably one or two groups independently of each other halo, hydroxy, unsubstituted lower alkyl or unsubstituted lower alkoxy groups, aryloxy optionally substituted with one or more, preferably one or two groups independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more, preferably one or two groups independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and nitrogen atoms of the group being optionally substituted with one or more, preferably one or two groups independently of each other halo, hydroxy, unsubstituted lower alkyl or unsubstituted lower alkoxy groups, 5- or 6-member heterocyclic group having from 1 to 3 heteroatoms selected from the group consisting of nitrogen, oxygen and sulfur, the carbon and nitogen (if present)atoms in the group being optionally substituted with one or more, preferably one or two groups independently of each other halo, hydroxy, unsubstituted lower alkyl or unsubstituted lower alkoxy groups, mercapto,(unsubstituted lower alkyl)thio, arylthio optionally substituted with one or more, preferably one or two groups independently of each other halo, hydroxy, unsubstituted lower alkyl or unsubstituted lower alkoxy groups, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)—, and —NR₁₃R₁₄ are as defined above.

“Alkenyl” refers to a lower alkyl group, as defined herein, consisting of at least two carbon atoms and at least one carbon-carbon double bond. Representative examples include, but are not limited to, ethenyl, 1-propenyl, 2-propenyl, 1-, 2-, or 3-butenyl, and the like.

“Alkynyl” refers to a lower alkyl group, as defined herein, consisting of at least two carbon atoms and at least one carbon-carbon triple bond. Representative examples include, but are not limited to, ethynyl, 1-propynyl, 2-propynyl, 1-, 2-, or 3-butynyl, and the like.

“Aryl” refers to an all-carbon monocyclic or fused-ring polycyclic (i.e., rings which share adjacent pairs of carbon atoms) groups of 1 to 12 carbon atoms having a completely conjugated pi-electron system. Examples, without limitation, of aryl groups are phenyl, naphthalenyl and anthracenyl. The aryl group may be substituted or unsubstituted. When substituted, the substituted group(s) is preferably one or more, more preferably one, two or three, even more preferably one or two, independently selected from the group consisting of unsubstituted lower alkyl, trihaloalkyl, halo, hydroxy, unsubstituted lower alkoxy, mercapto,(unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)—, and —NR₁₃R₁₄, with R₁₃ and R₁₄ as defined above. Preferably, the aryl group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

“Heteroaryl” refers to a monocyclic or fused ring (i.e., rings which share an adjacent pair of atoms) group of 5 to 12 ring atoms containing one, two, or three ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine and carbazole. The heteroaryl group may be substituted or unsubstituted. When substituted, the substituted group(s) is preferably one or more, more preferably one, two, or three, even more preferably one or two, independently selected from the group consisting of unsubstituted lower alkyl, trihaloalkyl, halo, hydroxy, unsubstituted lower alkoxy, mercapto,(unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)—, and —NR₁₃R₁₄, with R₁₃ and R₁₄ as defined above. Preferably, the heteroaryl group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

“Heterocyclic” refers to a monocyclic or fused ring group having in the ring(s) of 5 to 9 ring atoms in which one or two ring atoms are heteroatoms selected from N, O, or S(O)n (where n is an integer from 0 to 2), the remaining ring atoms being C. The rings may also have one or more double bonds. However, the rings do not have a completely conjugated pi-electron system. Examples, without limitation, of unsubstituted heterocyclic groups are pyrrolidino, piperidino, piperazino, morpholino, thiomorpholino, homopiperazino, and the like. The heterocyclic ring may be substituted or unsubstituted. When substituted, the substituted group(s) is preferably one or more, more preferably one, two or three, even more preferably one or two, independently selected from the group consisting of unsubstituted lower alkyl, trihaloalkyl, halo, hydroxy, unsubstituted lower alkoxy, mercapto,(unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)—, and —NR₁₃R₁₄, with R₁₃ and R₁₄ as defined above. Preferably, the heterocyclic group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

Preferably, the heterocyclic group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

“Hydroxy” refers to an —OH group.

“Alkoxy” refers to both an —O-(unsubstituted alkyl) and an —O-(unsubstituted cycloalkyl) group. Representative examples include, but are not limited to, e.g., methoxy, ethoxy, propoxy, butoxy, cyclopropyloxy, cyclobutyloxy, cyclopentyloxy, cyclohexyloxy, and the like.

“Aryloxy” refers to both an —O-aryl and an —O-heteroaryl group, as defined herein. Representative examples include, but are not limited to, phenoxy, pyridinyloxy, furanyloxy, thienyloxy, pyrimidinyloxy, pyrazinyloxy, and the like, and derivatives thereof.

“Mercapto” refers to an —SH group.

“Alkylthio” refers to both an —S-(unsubstituted alkyl) and an —S-(unsubstituted cycloalkyl) group. Representative examples include, but are not limited to, e.g., methylthio, ethylthio, propylthio, butylthio, cyclopropylthio, cyclobutylthio, cyclopentylthio, cyclohexylthio, and the like.

“Arylthio” refers to both an -S-aryl and an -S-heteroaryl group, as defined herein. Representative examples include, but are not limited to, phenylthio, pyridinylthio, furanylthio, thienylthio, pyrimidinylthio, and the like and derivatives thereof.

“Acyl” refers to a —C(O)—R″ group, where R″ is selected from the group consisting of hydrogen, unsubstituted lower alkyl, trihalomethyl, unsubstituted cycloalkyl, aryl optionally substituted with one or more, preferably one, two, or three substituents selected from the group consisting of unsubstituted lower alkyl, trihalomethyl, unsubstituted lower alkoxy, halo and —NR₁₃R₁₄, with R₁₃ and R₁₄ defined above (bonded through a ring carbon) optionally substituted with one or more, preferably one, two, or three substituents selected from the group consisting of unsubstituted lower alkyl, trihaloalkyl, unsubstituted lower alkoxy, halo and —NR₁₃R₁₄ groups and heterocyclic (bonded through a ring carbon) optionally substituted with one or more, preferably one, two, or three substituents selected from the group consisting of unsubstituted lower alkyl, trihaloalkyl, unsubstituted lower alkoxy, halo and

—NR₁₃R₁₄ groups. Representative acyl groups include, but are not limited to, acetyl, trifluoroacetyl, benzoyl, and the like.

“Aldehyde” refers to an acyl group in which R″ is hydrogen.

“Thioacyl” refers to a —C(S)—R″ group, with R″ as defined herein.

“Ester” refers to a —C(O)—R″ group with R″ as defined herein except that R″ cannot be hydrogen.

“Acetyl” group refers to a —C(O)CH₃ group.

“Halo” group refers to fluorine, chlorine, bromine or iodine, preferably fluorine or chlorine.

“Trihalomethyl” group refers to a —CX₃ group wherein X is a halo group as defined herein.

“Methylenedioxy” refers to a —OCH₂O— group where the two oxygen atoms are bonded to adjacent carbon atoms.

“Ethylenedioxy” group refers to a —OCH₂CH₂O— where the two oxygen atoms are bonded to adjacent carbon atoms.

“S-sulfonamido” refers to a —S(O)₂NR₁₃R₁₄ group, with R₁₃ and R₁₄ as defined herein.

“N-sulfonamido” refers to a —NR₁₃S(O)₂R group, with R₁₃ and R as defined herein.

“O-carbamyl” group refers to a —OC(O)NR₁₃R₁₄ group with R₁₃ and R₁₄ as defined herein.

“N-carbamyl” refers to an ROC(O)NR₁₄— group, with R and R₁₄ as defined herein.

“O-thiocarbamyl” refers to a —OC(S)NR₁₃R₁₄ group with R₁₃ and R₁₄ as defined herein.

“N-thiocarbamyl” refers to a ROC(S)NR₁₄— group, with R and R₁₄ as defined herein.

“Amino” refers to an —NR₁₃R₁₄ group, wherein R₁₃ and R₁₄ are both hydrogen.

“C-amido” refers to a —C(O)NR₁₃R₁₄ group with R₁₃ and R₁₄ as defined herein.

“N-amido” refers to a RC(O)NR₁₄— group, with R and R₁₄ as defined herein.

“Nitro” refers to a —NO₂ group.

“Haloalkyl” means an unsubstituted alkyl, preferably unsubstituted lower alkyl as defined above that is substituted with one or more same or different halo atoms, e.g., —CH₂Cl, —CF₃, —CH₂CF₃, —CH₂CCl₃, and the like.

“Aralkyl” means unsubstituted alkyl, preferably unsubstituted lower alkyl as defined above which is substituted with an aryl group as defined above, e.g., —CH₂phenyl, —(CH₂)₂phenyl, —(CH₂)₃phenyl, CH₃CH(CH₃)CH₂phenyl, and the like and derivatives thereof.

“Heteroaralkyl” group means unsubstituted alkyl, preferably unsubstituted lower alkyl as defined above which is substituted with a heteroaryl group, e.g., —CH₂pyridinyl, —(CH₂)₂pyrimidinyl, —(CH₂)₃imidazolyl, and the like, and derivatives thereof.

“Monoalkylamino” means a radical —NHR′ where R′ is an unsubstituted alkyl or unsubstituted cycloalkyl group as defined above, e.g., methylamino, (1-methylethyl)amino, cyclohexylamino, and the like.

“Dialkylamino” means a radical —NR′R′ where each R′ is independently an unsubstitued alkyl or unsubstituted cycloalkyl group as defined above, e.g., dimethylamino, diethylamino, (1-methylethyl)-ethylamino, cyclohexylmethylamino, cyclopentylmethylamino, and the like.

“Cyanoalkyl” means unsubstituted alkyl, preferably unsubstituted lower alkyl as defined above, which is substituted with 1 or 2 cyano groups.

“Optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocyclo group is not substituted with the alkyl group.

A “pharmaceutical composition” refers to a mixture of one or more of the compounds described herein, or physiologically/pharmaceutically acceptable salts or prodrugs thereof, with other chemical components, such as physiologically/pharmaceutically acceptable carriers and excipients. The purpose of a pharmaceutical composition is to facilitate administration of a compound to an organism.

The compound of Formula (I) may also act as a prodrug. A “prodrug” refers to an agent which is converted into the parent drug in vivo. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but then is metabolically hydrolyzed to the carboxylic acid, the active entity, once inside the cell where water solubility is beneficial.

A further example of a prodrug might be a short polypeptide, for example, without limitation, a 2-10 amino acid polypeptide, bonded through a terminal amino group to a carboxy group of a compound of this invention wherein the polypeptide is hydrolyzed or metabolized in vivo to release the active molecule. The prodrugs of a compound of Formula (I) are within the scope of this invention.

Additionally, it is contemplated that a compound of Formula (I) would be metabolized by enzymes in the body of the organism such as human being to generate a metabolite that can modulate the activity of the protein kinases. Such metabolites are within the scope of the present invention.

As used herein, a “physiologically/pharmaceutically acceptable carrier” refers to a carrier or diluent that does not cause significant irritation to an organism and does not abrogate the biological activity and properties of the administered compound.

An “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

As used herein, the term “pharmaceutically acceptable salt” refers to those salts which retain the biological effectiveness and properties of the parent compound. Such salts include:

(i) acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid such as the L-malate salt of 5-(5-fluoro-2-oxo-1,2-dihydroindol-3-ylidenemethyl)-2,4-dimethyl-1H-pyrrole-3-carboxylic acid(2-diethylaminoethyl)amide; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Method” refers to manners, means, techniques and procedures for accomplishing a given task including, but not limited to, those manners, means, techniques and procedures either known to, or readily developed from known manners, means, techniques and procedures by, practitioners of the chemical, pharmaceutical, biological, biochemical and medical arts.

“In vivo” refers to procedures performed within a living organism such as, without limitation, a mouse, rat or rabbit.

“Treat”, “treating” and “treatment” refer to a method of alleviating or abrogating cancer which may be treatable by administration of a compound of Formula (I) in combination with another chemotherapeutic agent. The term “treat” simply mean that the life expectancy of an individual affected with cancer will be increased or that one or more of the symptoms of the disease will be reduced.

“Cancer” refers to all forms of cancer, in particular, colon cancer, small cell lung cancer and breast cancer which includes all forms thereof.

“Patient” refers to any living entity comprised of at least one cell. A living organism can be as simple as, for example, a single eukariotic cell or as complex as a mammal, including a human being.

“Therapeutically effective amount” refers to that amount of the compounds (Formula I and the additional chemotherapeutic agent) being administered which will prevent, alleviate, ameliorate or relieve to some extent, one or more of the symptoms of the disorder being treated. In reference to the treatment of cancer, a therapeutically effective amount refers to that amount which has the effect of:

(1) reducing the size of the tumor;

(2) inhibiting (that is, slowing to some extent, preferably stopping) tumor metastasis;

(3) inhibiting to some extent (that is, slowing to some extent, preferably stopping) tumor growth,

(4) reducing blast cell counts, and/or

(5) relieving to some extent (or, preferably, eliminating) one or more symptoms associated with the cancer.

An enhanced therapeutic effect refers to an effect of the combination that exceeds the effect of either drug alone.

### ADMINISTRATION AND PHARMACEUTICAL COMPOSITION

The claimed methods involve administration of a compound of formula I or a pharmaceutically acceptable salt thereof in combination with an additional chemotherapeutic agent, to a human patient. Alternatively, the compounds of Formula I in combination with an additional chemotherapeutic agent can be administered in pharmaceutical compositions in which the foregoing materials are mixed with suitable carriers or excipient(s). Techniques for formulation and administration of drugs may be found in “Remington's Pharmacological Sciences,” Mack Publishing Co., Easton, Pa., latest edition.

As used herein, “administer” or “administration” refers to the delivery of a compound of Formula (I) or a pharmaceutically acceptable salt thereof in combination with an additional chemotherapeutic agent or of a pharmaceutical composition containing a compound of Formula (I) in combination with an additional chemotherapeutic agent or a pharmaceutically acceptable salt thereof of this invention to an organism for the purpose of treatment of cancer. With respect to the additional chemotherapeutic agents, doses and modes of administration involve standard protocols which are understood and practiced by those having ordinary skill in the art.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. The preferred routes of administration are oral and parenteral.

Alternatively, one may administer the compound in a local rather than systemic manner, for example, via injection of the compound directly into a solid tumor, often in a depot or sustained release formulation.

Furthermore, one may administer the drug in a targeted drug delivery system, for example, in a liposome coated with tumor-specific antibody. The liposomes will be targeted to and taken up selectively by the tumor.

Processes well known in the art, e.g., by means of conventional mixing, dissolving, granulating, dragee-making, levigating, emulsifying, encapsulating, entrapping or lyophilizing processes may manufacture pharmaceutical compositions of the present invention.

Pharmaceutical compositions for use in accordance with the present invention may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also.

Pharmaceutical compositions which may also be used include hard gelatin capsules. As a non-limiting example, compound 1 in a capsule oral drug product formulation may be as 50 and 200 mg dose strengths. The two dose strengths are made from the same granules by filling into different size hard gelatin capsules, size 3 for the 50 mg capsule and size 0 for the 200 mg capsule. Determination of the protocol for combination therapy is well within the ordinary skill of the practicing physician and is determined by the particular disease state and the state of the patient and chemotherapeutic regimen received by the patient.

The capsules may be packaged into brown glass or plastic bottles to protect the active compound from light. The containers containing the active compound capsule formulation must be stored at controlled room temperature (15-30° C.).

For administration by inhalation, the compounds for use according to the present invention are conveniently delivered in the form of an aerosol spray using a pressurized pack or a nebulizer and a suitable propellant, e.g., without limitation, dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

In addition to the fomulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharamcologically acceptable oil), with ion exchange resins, or as a sparingly soluble derivative such as, without limitation, a sparingly soluble salt.

A non-limiting example of a pharmaceutical carrier for the hydrophobic compounds of the invention is a cosolvent system comprising benzyl alcohol, a nonpolar surfactant, a water-miscible organic polymer and an aqueous phase such as the VPD co-solvent system. VPD is a solution of 3% w/v benzyl alcohol, 8% w/v of the nonpolar surfactant Polysorbate 80, and 65% w/v polyethylene glycol 300, made up to volume in absolute ethanol. The VPD co-solvent system (VPD:D5W) consists of VPD diluted 1:1 with a 5% dextrose in water solution. This co-solvent system dissolves hydrophobic compounds well, and itself produces low toxicity upon systemic administration. Naturally, the proportions of such a co-solvent system may be varied considerably without destroying its solubility and toxicity characteristics. Furthermore, the identity of the co-solvent components may be varied: for example, other low-toxicity nonpolar surfactants may be used instead of Polysorbate 80, the fraction size of polyethylene glycol may be varied, other biocompatible polymers may replace polyethylene glycol, e.g., polyvinyl pyrrolidone, and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions herein also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the compounds of the Formula I may be provided as physiologically acceptable salts wherein the compound may form the negatively or the positively charged species. Examples of salts in which the compound forms the positively charged moiety include, without limitation, quaternary ammonium, salts such as the hydrochloride, sulfate, carbonate, lactate, tartrate, malate, maleate, succinate wherein the nitrogen atom of the quaternary ammonium group is a nitrogen of the selected compound of this invention which has reacted with the appropriate acid. Salts in which a compound of this invention forms the negatively charged species include, without limitation, the sodium, potassium, calcium and magnesium salts formed by the reaction of a carboxylic acid group in the compound with an appropriate base (e.g. sodium hydroxide (NaOH), potassium hydroxide (KOH), Calcium hydroxide (Ca(OH)₂), etc.).

Pharmaceutical compositions suitable for use in the present invention include compositions wherein the active ingredients are contained in an amount sufficient to achieve the intended purpose, e.g., treatment of cancer patients.

More specifically, a “therapeutically effective amount” means an amount of compound effective to prevent, alleviate or ameliorate symptoms of cancer or prolong the survival of the subject being treated.

Determination of a therapeutically effective amount is well within the capability of those skilled in the art, especially in light of the detailed disclosure provided herein.

For any compound used in the methods of the invention, the therapeutically effective amount or dose can be estimated initially from cell culture assays. Then, the dosage can be formulated for use in animal models so as to achieve a circulating concentration range that includes the IC50 as determined in cell culture (i.e., the concentration of the test compound which achieves a half-maximal inhibition of phosphorylation of the target receptor tyrosine kinase). Such information can then be used to more accurately determine useful doses in humans.

Toxicity and therapeutic efficacy of the compounds described herein can be determined by standard pharmaceutical procedures in cell cultures or experimental animals, e.g., by determining the IC50 and the LD50, wherein the LD50 is the concentration of test compound which achieves a half-maximal inhibition of lethality, for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma levels of the active species which are sufficient to maintain the kinase modulating effects. These plasma levels are referred to as minimal effective concentrations (MECs). The MEC will vary for each compound but can be estimated from in vitro data, e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%.

At present, the therapeutically effective amounts of compounds of Formula (I) may range from approximately 25 mg/m² to 1500 mg/m² per day; preferably about 3 mg/m2/day. Even more preferably 50 mg/qm qd till 400 mg/qd. The therapeutically effective amount of the additional chemotherapeutic agent is adminstered to the patient based on recommendations of the manufacturer. However, the two agents in combination may allow for lower doses of the additional chemotherapeutic agent to be administered.

In cases of local administration or selective uptake, the effective local concentration of the drug may not be related to plasma concentration and other procedures known in the art may be employed to determine the correct dosage amount and interval.

The amount of a composition administered will, of course, be dependent on the subject being treated, the severity of the affliction, the manner of administration, the judgment of the prescribing physician, etc.

It is contemplated that the inventive method could be used in combination with other cancer therapies, bone marrow transplantation and hormone therapy.

Finally, it is also contemplated that the inventive combination could be further combined with, e.g., an antiangiogenic agent, such as, but not limited to a cyclooxygenase inhibitor such as celecoxib.

### General Synthetic Procedure

The following general methodology may be employed to prepare the compounds of this invention.

The appropriately substituted 2-oxindole (1 equiv.), the appropriately substituted aldehyde (1.2 equiv.) and a base (0.1 equiv.) are mixed in a solvent (1-2 mL/mmol 2-oxindole) and the mixture is then heated for from about 2 to about 12 hours. After cooling, the precipitate that forms is filtered, washed with cold ethanol or ether and vacuum dried to give the solid product. If no precipitate forms, the reaction mixture is concentrated and the residue is triturated with dichloromethane/ether, the resulting solid is collected by filtration and then dried. The product may optionally be further purified by chromatography.

The base may be an organic or an inorganic base. If an organic base is used, preferably it is a nitrogen base. Examples of organic nitrogen bases include, but are not limited to, diisopropylamine, trimethylamine, triethylamine, aniline, pyridine, 1,8-diazabicyclo[5.4.1 ]undec-7-ene, pyrrolidine and piperidine.

Examples of inorganic bases are, without limitation, ammonia, alkali metal or alkaline earth hydroxides, phosphates, carbonates, bicarbonates, bisulfates and amides. The alkali metals include, lithium, sodium and potassium while the alkaline earths include calcium, magnesium and barium.

In a presently preferred embodiment of this invention, when the solvent is a protic solvent, such as water or alcohol, the base is an alkali metal or an alkaline earth inorganic base, preferably, a alkali metal or an alkaline earth hydroxide.

It will be clear to those skilled in the art, based both on known general principles of organic synthesis and on the disclosures herein which base would be most appropriate for the reaction contemplated.

The solvent in which the reaction is carried out may be a protic or an aprotic solvent, preferably it is a protic solvent. A “protic solvent” is a solvent which has hydrogen atom(s) covalently bonded to oxygen or nitrogen atoms which renders the hydrogen atoms appreciably acidic and thus capable of being “shared” with a solute through hydrogen bonding. Examples of protic solvents include, without limitation, water and alcohols.

An “aprotic solvent” may be polar or non-polar but, in either case, does not contain acidic hydrogens and therefore is not capable of hydrogen bonding with solutes. Examples, without limitation, of non-polar aprotic solvents, are pentane, hexane, benzene, toluene, methylene chloride and carbon tetrachloride. Examples of polar aprotic solvents are chloroform, tetrahydro-furan, dimethylsulfoxide and dimethylformamide.

In a presently preferred embodiment of this invention, the solvent is a protic solvent, preferably water or an alcohol such as ethanol.

The reaction is carried out at temperatures greater than room temperature. The temperature is generally from about 30° C. to about 150° C., preferably about 80° C. to about 100° C., most preferable about 75° C. to about 85° C., which is about the boiling point of ethanol. By “about” is meant that the temperature range is preferably within 10 degrees Celsius of the indicated temperature, more preferably within 5 degrees Celsius of the indicated temperature and, most preferably, within 2 degrees Celsius of the indicated temperature. Thus, for example, by “about 75° C.” is meant 75° C.±10° C., preferably 75° C.±5° C. and most preferably, 75° C.±2° C.

2-Oxindoles and aldehydes, may be readily synthesized using techniques well known in the chemical arts. It will be appreciated by those skilled in the art that other synthetic pathways for forming the compounds of the invention are available and that the following is offered by way of example and not limitation.

Compounds of the present invention can be prepared according to the following methodologies and as described, e.g., in U.S. Pat. No. 6,573,293, WO 01/60814, WO 00/08202, U.S. patent Publication No. 2003/0069298, WO 03/016305, U.S. patent application Ser. No. 10/367,008, filed Feb. 14, 2003, U.S. Pat. No. 6,642,232, and U.S. patent application Ser. No. 10/076,140, filed Feb. 15, 2002, all of which are incorporated by reference in their entirety.

Preferred formulations are described in U.S. patent application Ser. No. 10/658,801, filed Sep. 10. 2003, the disclosure of which is incorporated herein by reference.

### Synthetic Methodologies

### Method A: Formylation of pyrroles

POCl₃ (1.1 equiv.) is added dropwise to dimethylformamide (3 equiv.)at −10° C. followed by addition of the appropriate pyrrole dissolved in dimethylformamide. After stirring for two hours, the reaction mixture is diluted with H₂O and basified to pH 11 with 10 N KOH. The precipitate which forms is collected by filtration, washed with H₂O and dried in a vacuum oven to give the desired aldehyde.

### Method B: Saponification of pyrrolecarboxylic acid esters

A mixture of a pyrrolecarboxylic acid ester and KOH (2-4 equiv.) in EtOH is refluxed until reaction completion is indicated by thin layer chromatography (TLC). The cooled reaction mixture is acidified to pH 3 with 1 N HCl. The precipitate which forms is collected by filtration, washed with H₂O and dried in a vacuum oven to give the desired pyrrolecarboxylic acid.

### Method C: Amidation

To a stirred solution of a pyrrolecarboxylic acid dissolved in dimethylformamide(0.3M) is added 1-ethyl-3-(3-dimethylamino-propyl)carbodiimide (1.2 equiv.), 1-hydroxybenzotriazole (1.2 equiv.), and triethylamine (2 equiv.). The appropriate amine is added (1 equiv.) and the reaction stirred until completion is indicated by TLC. Ethyl acetate is then added to the reaction mixture and the solution washed with saturated NaHCO₃ and brine (with extra salt), dried over anhydrous MgSO₄ and concentrated to afford the desired amide.

### Method D: Condensation of aldehydes and oxindoles Containing carboxylic acid Substituents

A mixture of the oxindole (1 equivalent), 1 equivalent of the aldehyde and 1-3 equivalents of piperidine (or pyrrolidine) in ethanol (0.4 M) is stirred at 90-100° C. until reaction completion is indicated by TLC. The mixture is then concentrated and the residue acidified with 2N HCl. The precipitate that forms is washed with H₂O and EtOH and then dried in a vacuum oven to give the product.

### Method E: Condensation of aldehydes and oxindoles not Containing carboxylic acid Substituents

A mixture of the oxindole (1 equivalent), 1 equivalent of the aldehyde and 1-3 equivalents of piperidine (or pyrrolidine) in ethanol (0.4 M) is stirred at 90-100° C. until reaction completion is indicated by TLC. The mixture is cooled to room temperature and the solid which forms is collected by vacuum filtration, washed with ethanol and dried to give the product. If a precipitate does not form upon cooling of the reaction mixture, the mixture is concentrated and purified by column chromatography.

The following examples are given to illustrate the present invention. It should be understood, however, that the invention is not to be limited to the specific conditions or details described in these examples. Throughout the specification, any and all references to a publicly available documents are specifically incorporated into this patent application by reference.

### SYNTHETIC EXAMPLES

### Example 1

### Synthesis of (3Z)-3-{[3,5-dimethyl-4-(morpholin-4-yl)piperidin-1-ylcarbonyl]-1H-pyrrol-2-ylmethylidene}-5-fluoro-1,3-dihydro-2H-indol-2-one (Compound 9)

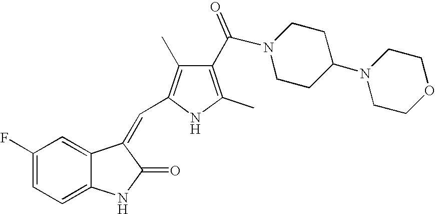

Step 1: To a stirred mixture of 4-amino-1-benzylpiperidine (Aldrich, 1.53 mL, 7.5 mmol), K₂CO₃ (2.28 g, 16.5 mmol), and DMF (15 mL) heated at 50° C. was added dropwise over 60 min bis(2-bromoethyl)ether (Aldrich, tech. 90%, 0.962 mL, 7.65 mmol). After stirring 6 h at 80° C., TLC (90:10:1 chloroform/MeOH/aq. conc NH₄OH) indicated formation of a new spot. Heating was continued as the solvent was evaporated by blowing with a stream of nitrogen over 2 h. The crude material was relatively pure, but subjected to a relatively short silica gel column (1% to 6% gradient of 9:1 MeOH/aq. NH₄OH in chloroform). Evaporation of the pure fractions gave ˜1.7 g of the diamine 4-(morpholin-4-yl)-1-benzylpiperidine as a waxy solid.

¹HNMR (400 MHz, d₆-DMSO) δ 7.31 (m, 4H), 7.26 (m 1H), 3.72 (t, J=4.7 Hz, 4H), 3.49 (s, 2H), 2.94 (br d, J=5.9 Hz, 2H), 2.54 (t, J=4.7 Hz, 4H), 2.19 (tt, J=11.5, 3.9 Hz, 1H), 1.96 (td, J=11.7, 2.2 Hz, 2H), 1.78 (br d, J=12.5 Hz, 2H), 1.55 (m, 2H).

Step 2: A stirred mixture of Pd(OH)₂ (20% on carbon (<50% wet), 390 mg, 25 wt %), methanol (50 mL), and ≦1.7 M HCl (3 eq, ˜10.6 mL—including water added later when ppt was seen) under nitrogen was exchanged to 1 atm. hydrogen atmosphere by flushing (˜20 s) using a balloon of nitrogen into the vessel and out through an oil bubbler. After 20 min. the reaction mixture under hydrogen was heated to 50° C. and 4-(morpholin-4-yl)-1-benzylpiperidine (1.56 g, 6.0 mmol) in methanol (8 mL) was added dropwise over 30 min. After 10 h, tlc indicated all starting amine was consumed to a more polar spot (ninhydrin active). The reaction mixture was then filtered through Celite and evaporated to yield the 4-(morpholin-4-yl)piperidine dihydrochloride as an off-white solid. This material was subjected to free-basing using excess basic resin (>16 g, Bio-Rad Laboratories, AG 1-X8, 20-50 mesh, hydroxide form, methanol washed two times) and a methanol mixture of the amine hydrochloride. After swirling with the resin for 30 min., the methanol solution was decanted and evaporated to yield 932 mg of 4-(morpholin-4-yl)piperidine free base as a waxy crystalline solid.

¹HNMR (400 MHz, d₆-DMSO) δ 3.53 (br s, 4H), 3.30 (v br s, 1H(+H₂O)), 2.92 (br d, J=11.7 Hz, 1H), 2.41 (s, 4H), 2.35 (˜obscd t, J=11.7 Hz, 2H), 2.12 (br t, 1H), 1.65 (br d, J=11.7 Hz, 2H), 1.18 (br q, J=10.9 Hz, 2H); LCMS-APCI m/z 171 [M+1]⁺.

Step 3: (3Z)-3-(3,5-Dimethyl-4-carboxy-1H-pyrrol-2-ylmethylidene)-5-fluoro-1,3-dihydro-2H-indol-2-one (120 mg, 0.40 mmol), prepared as described in PCT Publication No 01/60814, and BOP (221 mg, 0.50 mmol) were suspended in DMF (5 mL) with good stirring at room temperature and triethylamine (134 μL, 0.96 mmol) was added. After 10-15 min., to the homogeneous reaction mixture was added the 4-(morpholin-4-yl)piperidine (85 mg, 0.50 mmol) all at once. The reaction mixture was stirred for 48 h (might be done much earlier), then transferred to a funnel containing chloroform-isopropanol (5/1) and 5% aq. LiCl. The cloudy-orange organic phase was separated, washed with additional 5% aq LiCl (2×), 1 M aq NaOH (3×), satd aq NaCl (1×), and then dried (Na₂SO₄) and evaporated to yield the crude product (96.3% pure; trace HMPA by ¹HNMR). This crude product was then further purified by passage through a very short column (3 cm) of silica gel (5 to 15% gradient of MeOH in DCM) where a trace of faster moving 3E-isomer was removed. The pure fractions were evaporated and recrystallized overnight from a satd EtOAc soln which was diluted with Et₂O (˜3-fold) and chilled at 0° C. The mother liquor was decanted to yield after full vacuum the desired compound as orange crystals (153 mg 85%).

¹HNMR (400 MHz, d₆-DMSO) δ 13.60 (s, 1H), 10.87 (s, 1H), 7.72 (dd, J=9.4, 2.7 Hz, 1H), 7.68 (s, 1H), 6.91 (td, J=9.3, 2.6 Hz, 1H), 6.82 (dd, J=8.6, 4.7 Hz, 1H), 3.54 (app br t, J=4.3 Hz, 4H), 3.31 (2× s, 3H+3H), 2.43 (br s, 4H), 2.36 (m, 1H), 2.25 (br m, 6H), 1.79 (br s, 2H), 1.22 (br s, 2H); LCMS m/z 453 [M+1]⁺.

Proceeding as described in Example 1 but substituting (3Z)-3-(3,5-dimethyl-4-carboxy-1H-pyrrol-2-ylmethylidene)-5-fluoro-1,3-dihydro-2H-indol-2-one for (3Z)-3-(3,5-dimethyl-4-carboxy-1H-pyrrol-2-ylmethylidene)-1,3-dihydro-2H-indol-2-one gave (3Z)-3-{[3,5-dimethyl-4-(morpholin-4-yl)piperidin-1-ylcarbonyl]-1H-pyrrol-2-ylmethylidene}-, 3-dihydro-2H-indol-2-one. ¹HNMR (400 MHz, d₆-DMSO) δ 13.55 (s, 1H), 10.87 (s, 1H), 7.74 (d, J=7.6 Hz, 1H), 7.59 (s, 1H), 7.11 (t, J=7.6 Hz, 1H), 6.97 (t, J=7.6 Hz, 1H), 6.86 (d, J=7.4 Hz, 1H), 3.54 (app br t, J=4.3 Hz, 4H), 3.31 (2× s, 3H+3H), 2.43 (br s, 4H), 2.35 (m, 1H), 2.28 (br m, 6H), 1.79 (br s, 2H), 1.22 (br s, 2H); LCMS m/z 435 [M+1]⁺.

Proceeding as described in Example 1 but substituting (3Z)-3-(3,5-dimethyl-4-carboxy-1H-pyrrol-2-ylmethylidene)-5-fluoro-1,3-dihydro-2H-indol-2-one for (3Z)-3-(3,5-dimethyl-4-carboxy-1H-pyrrol-2-ylmethylidene)-5-chloro-1,3-dihydro-2H-indol-2-one gave (3Z)-3-{[3,5-dimethyl-4-(morpholin-4-yl)piperidin-1-ylcarbonyl]-1H-pyrrol-2-ylmethylidene}-5-chloro-1,3-dihydro-2H-indol-2-one.

¹HNMR (400 MHz, d₆-DMSO) δ 13.56 (s, 1H), 10.97 (s, 1H), 7.95 (d, J=2.0 Hz, 1H), 7.74 (s, 1H), 7.11 (dd, J=8.2, 2.0 Hz, 1H), 6.85 (d, J=8.2 Hz, 1H), 3.54 (app br t, J=˜4 Hz, 4H), 3.31 (2× s, 3H+3H), 2.43 (br s, 4H), 2.37 (m, 1H), 2.25 (br m, 6H), 1.79 (br s, 2H), 1.23 (br s, 2H); LCMS m/z 470 [M+1]⁺.

Proceeding as described in Example 1 but substituting 4-(morpholin-4-yl)-piperidine with commercially available 4-(1-pyrrolidinyl)-piperidine gave (3Z)-3-{[3,5-dimethyl-4-[4-(pyrrolidin-1-yl)piperidin-1-ylcarbonyl]-1H-pyrrol-2-yl)methylidene]-5-fluoro-1,3-dihydro-2H-indol-2-one.

¹HNMR (400 MHz, d₆-DMSO) δ E/Z isomer mixture; LCMS m/z 437 [M+1]⁺.

Synthesis of the above examples can proceed according to the procedure of U.S. U.S. patent Publication No. 2003/0130280, incorporated by reference in its entirety.

### Example 2

### Synthesis of (3Z)-3-{[3,5-dimethyl-4-(morpholin-4-yl)azetidin-1-ylcarbonyl]-1H-pyrrol-2-ylmethylidene}-5-fluoro-1,3-dihydro-2H-indol-2-one

Step 1: A solution of 1-azabicyclo[1.1.0]butane, prepared from 2,3-dibromopropylamine hydrobromide (58.8 mmol) according to a known procedure described in Tetrahedron Letters 40 (1999) 3761-64, was slowly added to a solution of morpholine (15.7 mL; 180 mmol) and sulfuric acid (3.3 g of 96% soln.) in anhydrous non-denaturated ethanol (250 mL) at 0° C. The reaction mixture was stirred on ice bath for 30 min., then at room temperature for 8 h. Calcium hydroxide (5.5 g) and 100 mL of water was added and the obtained slurry was stirred for 1 h and then filtered through a pad of Celite. The filtrate was concentrated and distilled at reduced pressure (20 mm Hg) to remove water and an excess of morpholine. The distillation residue was re-distilled at high vacuum using a Kugelrohr apparatus to obtain a pure 4-(azetidin-3-yl)morpholine in 33% yield (2.759 g) as a colorless oily liquid.

¹³C-NMR (CDCl₃, 100 MHz): 66.71(2C), 59.37 (1C), 51.46 (2C), 49.95(2C) ¹H (CDCl₃, 400 MHz): 3.727 (t, J=4.4 Hz, 4H), 3.619 (t, J=8 Hz, 2H), 3.566 (t, J=8 Hz, 2H), 3.227 (m, J=7 Hz, 1H), 2.895 (br s, 1H), 2.329 (br s, 4H)

Step 2: 1-(8-Azabenztriazolyl)-ester of (3Z)-3-({3,5-dimethyl-4-carboxy]1-H-pyrrol-2-yl}methylene)-5-fluoro-1.3-dihydro-2H-indol-2-one (0.5 mmol, 210 mg) [prepared by activating (3Z)-3-(3,3-dimethyl-4-carboxy-1-H-pyrrol-2-ylmethylene)-5-fluoro-1.3-dihydro-2H-indol-2-one (480 mg; 1.6 mmol) with the HATU reagent (570 mg, 1.5 mmol) in the presence of Hunig base (3.0 mmol, 0.525 mL) in DMF (5 mL) and isolated in pure form by precipitation with chloroform (5 mL) and drying on high vacuum in 92% yield (579 mg)] was suspended in anhydrous DMA (1.0 mL). A solution of 4-(azetidin-3-yl)-morpholine; (142.5 mg, 1 mmol) in anhydrous DMA (1.0 mL) was added in one portion and the obtained solution was stirred at room temperature for 20 min. The reaction mixture was evaporated at room temperature using an oil pump, the thick residue was diluted with 6 mL of a mixture of methanol plus diethyl amine (20:1; v/v), inoculated mechanically and placed into a refrigerator (+3° C.) for 8 hours. The precipitates were filtered (with a brief wash with an ice-cold methanol) and dried on high vacuum to give the desired product. 71.5% yield (152 mg of an orange solid).

LC/MS: +APCI: M+1=425; −APCI: M−1=423. ¹⁹F-NMR (d-DMSO, 376.5 MHz): −122.94 (m, 1F). ¹H (d-DMSO, 400 MHz): 13.651 (s, 1H), 10.907 (s, 1H), 7.754 (dd, J=9.4 Hz, J=2.4 Hz, 1H), 7.700 (s, 1H), 6.935 (dt, J=8.2 Hz, J=2.4 Hz, 1H), 6.841 (dd, J=8.6 Hz, J=3.9 Hz; 1H), 3.963 (br s, 2H), 3.793 (br s, 2H), 3.581 (br t, J=4.3 Hz, 4H), 3.133 (m, 1H), 2.367 (s, 3H), 2.340 (s, 3H), 2.295 (br s, 4H).

Proceeding as described in Example 2 but substituting (3Z)-3-(3,5-dimethyl-4-carboxy-1H-pyrrol-2-ylmethylidene)-5-fluoro-1,3-dihydro-2H-indol-2-one with (3Z)-3-(3,5-dimethyl-4-carboxy-1H-pyrrol-2-ylmethylidene)-5-chloro-1,3-dihydro-2H-indol-2-one gave (3Z)-3-{[3,5-dimethyl-4-(morpholin-4-yl)azetidin-1-ylcarbonyl]-1H-pyrrol-2-ylmethylidene}-5-chloro-1,3-dihydro-2H-indol-2-one as an orange solid.

LC/MS: +APCI: M+1=441; −APCI: M−1=440,441. ¹H (d-DMSO, 400 MHz): 13.607 (s, 1H), 11.006 (s,1H), 7.976 (d, J=2.0 Hz, 1H), 7.756 (s, 1H), 7.136 (dd, J=8.2 Hz, J=2.0 Hz, 1H), 6.869 (d, J=8.2 Hz, 1H), 3.964 (br s, 2H), 3.793 (br s, 2H), 3.582 (br t, J=4.3 Hz, 4H), 3.134 (m,1H), 2.369 (s, 3H), 2.347 (s, 3H), 2.296 (br s, 4H).

Proceeding as described in Example 2 but substituting 4-(azetidin-3-yl)morpholine with 4-(azetidin-3-yl)-cis-3,5-dimethylmorpholine (prepared in a procedure analogous to the preparation of 4-(azetidin-3-yl)-morpholine but using cis-3,5-dimethylmorpholine (20.7 g; 180 mmol) in place of morpholine) gave (3Z)-3-{[3,5-dimethyl-4-(2,5-dimethylmorpholin-4-yl)azetidin-1-ylcarbonyl]-1H-pyrrol-2-ylmethylidene}-5-fluoro-1,3-dihydro-2H-indol-2-one as an orange solid.

LC/MS: +APCI: M+1=453; −APCI: M−1=451. ¹⁹F-NMR (d-DMSO, 376.5 MHz): -122.94 (m, 1F). ¹H (d-DMSO, 400 MHz): 13.651 (s, 1H), 10.907 (s; 1H), 7.758 (dd, J=9.4 Hz, J=2.3 Hz; 1H), 7.700 (s, 1H), 6.935 (dt, J=8.6 Hz, J=2.7 Hz, 1H), 6.842 (dd, J=8.2 Hz, J=4.3 Hz, 1H), 3.961 (br s, 2H), 3.790 (br s, 2H), 3.546 (br m, 2H), 3.092 (m, 1H), 2.690 (br s; 2H), 2.364 (s, 3H), 2.338 (s, 3H), 1.492 (br m, 2H), 1.038 (br s, 6H).

Proceeding as described in Example 2 but substituting (3Z)-3-(3,5-dimethyl-4-carboxy-1H-pyrrol-2-ylmethylidene)-5-fluoro-1,3-dihydro-2H-indol-2-one with (3Z)-3-(3,5-dimethyl-4-carboxy-1H-pyrrol-2-ylmethylidene)-5-chloro-1,3-dihydro-2H-indol-2-one and 4-(azetidin-3-yl)morpholine with 4-(azetidin-3-yl)-cis-3,5-dimethylmorpholine gave (3Z)-3-{[3,5-dimethyl-4-(3,5-dimethylmorpholin-4-yl)azetidin-1-ylcarbonyl]-1H-pyrrol-2-ylmethylidene}-5-chloro-1,3-dihydro-2H-indol-2-one as an orange solid.

LC/MS: +APCI: M+1=469, 470; −APCI: M−1=468,469. ¹H (d-DMSO, 400 MHz): 13.606 (s, 1H), 11.008 (s, 1H), 7.979 (d, J=2.0 Hz, 1H), 7.758 (s, 1H), 7.138 (dd, J=8.2 Hz, J=2.0 Hz, 1H), 6.870 (d, J=8.2 Hz, 1H), 3.964 (br s, 2H), 3.790 (br s, 2H), 3.547 (br m, 2H), 3.095 (m, 1H), 2.691 (br s, 2H), 2.366 (s, 3H), 2.345 (s, 3H), 1.494 (br m, 2H), 1.039 (br s, 6H).

Proceeding as described in Example 1 above, but substituting 4-(morpholin-4-yl)-piperidine with 2-(R)-pyrrolidin-1-ylmethylpyrrolidine prepared as described below provided (3Z)-3-{[3,5-dimethyl-2R-(pyrrolidin-1-ylmethyl)pyrrolidin-1-ylcarbonyl]-1H-pyrrol-2-ylmethylidene}-5-fluoro-1,3-dihydro-2H-indol-2-one.

### Synthesis of 2(R)-pyrrolidin-1-ylmethylpyrrolidine

Step 1: To a solution of (+)-Carbobenzyloxy-D-proline (1.5 g, 6.0 mmol), EDC (2.3 g, 12.0 mmol) and HOBt (800 mg, 12.9 mmol) in DMF (20 mL) was added trietylamine (1.5 mL) and pyrrolidine (1.0 mL, 12.0 mmol). It was stirred for 18 h at rt. Sat. NaHCO₃ was added, it was extracted with CH₂CL₂ (three times). The organic layers were separated and dried over Na₂SO₄. The solvent was removed and the residue was purified by silica gel chromatography (EtOAc) to give 1-(R)—[N-(benzyloxycarbonyl)-pyrolyl]pyrrolidine as a white solid (94%).

¹H NMR (400 MHz, CDCl₃, all rotamers) δ 1.57-1.66 (m, 1H), 1.71-2.02 (m, 5H), 2.04-2.19 (m, 2H), 3.26-3.43 (m, 3H), 3.44-3.78 (m, 3H), 4.41 (dd, J=4.5, 7.6 Hz, 0.5H), 4.52 (dd, J=3.7, 7.6 Hz, 0.5H), 4.99 (d, J=12.1 Hz, 0.5H), 5.05 (d, J=12.5 Hz, 0.5H), 5.13 (d, J=12.1 Hz, 0.5H), 5.20 (d, J=12.5 Hz, 0.5H), 7.27-7.38 (m, 5H).

Step 2: A mixture of 1-(R)—[N-(benzyloxycarbonyl)prolyl]pyrrolidine (2.7 g, 8.9 mmol) and 5% Pd—C catalyst (270 mg) in methanol (15 mL) were stirred under a hydrogen atmosphere for 20 h. The reaction mixture was filtered through celite and the solvent was removed yielding 2(R)-prolylpyrrolidine as a viscous oil (80%), which was used without further purification for the next step.

¹H NMR (400 MHz, d₆-DMSO) δ 1.52-1.78 (m, 5H), 1.82-1.89 (m, 2H), 1.97-2.04 (m, 1H), 2.63-2.71 (m, 1H), 2.97-3.02 (m, 1H), 3.22-3.35 (m, 3H), 3.48-3.54 (m, 1H), 3.72 (dd, J=6.1, 8.0 Hz, 1H).

Step 3: 2-(R)-Prolylpyrrolidine (1.2 g, 7.1 mmol) was dissolved in THF (10 mL). The reaction mixture was cooled to 0° C. and BH₃, 1M in THF (10 mL, 10 mmol) was dropwise at 0° C. The reaction mixture was refluxed for 16 h, 3 M HCl (4.7 mL). 2 M NaOH solution was added until pH 10 was reached. The product was extracted with 5% MeOH in CH₂Cl₂ (three times). The organic layers were dried over Na₂SO₄ and the solvent was removed to provide the title compound as a slightly yellow liquid (73%), which was used without further purification for the next step.

¹H NMR (400 MHz, d₆-DMSO) δ 1.22-1.30 (m, 1H), 1.55-1.69 (m, 6H), 1.71-1.79 (m, 1H), 2.26-2.30 (m, 1H), 2.33-2.38 (m, 1H), 2.40-2.45 (m, 4H), 2.65-2.71 (m, 1H), 2.78-2.84 (m, 1H), 3.02-3.09 (m, 1H).

Proceeding as described in Example 1 above, but substituting 4-(morpholin-4-yl)-piperidine with 2-(S)-pyrrolidin-1-ylmethylpyrrolidine (prepared as described above, by substituting (+)-carbobenzyloxy-D-proline with carbobenzyloxy-L-proline) provided (3Z)-3-{[3,5-dimethyl-2S-(pyrrolidin-1-ylmethyl)pyrrolidin-1-ylcarbonyl]-1H-pyrrol-2-ylmethylidene}-5-fluoro-1,3-dihydro-2H-indol-2-one.

### Example 3

### Synthesis of 5-[5-fluoro-2-oxo-1,2-dihydro-indol-(3Z)-ylidene-methyl]-2,4-dimethyl-1H-pyrrole-3-carboxylic acid

Step 1: Dimethylformamide (25 mL, 3 eq.) was cooled with stirring in an ice bath. To this was added POCl₃ (1.1 eq., 10.8 mL). After 30 minutes, a solution of the 3,5-dimethyl-4-ethylester pyrrole (17.7 g, 105.8 mmol) in DMF (2M, 40 mL) was added to the reaction and stirring continued. After 2 hour, the reaction was diluted with water (250 mL) and basified to pH=11 with 1N aqueous NaOH. The white solid was removed by filtration, rinsing with water and then hexanes and dried to afford 5-formyl-2,4-dimethyl-1H-pyrrole-3-carboxylic acid ethyl ester (19.75 g, 95%) as a tan solid.

¹H NMR (360 MHz, DMSO-d6) δ 12.11 (br s, 1H, NH), 9.59 (s, 1H, CHO), 4.17 (q, J=6.7 Hz, 2H, OCH₂CH₃), 2.44 (s, 3H, CH₃), 2.40 (s, 3H, CH₃), 1.26 )d, J=6.7 Hz, 3H, OCH₂CH₃).

Step 2: 5-Formyl-2,4-dimethyl-1H-pyrrole-3-carboxylic acid ethyl ester (2 g, 10 mmol) was added to a solution of potassium hydroxide (3 g, 53 mmol) dissolved in methanol (3 mL) and water (10 mL). The mixture was refluxed for 3 hours, cooled to room temperature and acidified with 6 N hydrochloric acid to pH 3. The solid was collected by filtration, washed with water and dried in a vacuum oven overnight to give 5-formyl-2,4-dimethyl-1H-pyrrole-3-carboxylic acid (1.6 g, 93%).

¹H NMR (300 MHz, DMSO-d6) δ 12.09 (s, br, 2H, NH & COOH), 9.59 (s, 1H, CHO), 2.44 (s, 3H, CH₃), 2.40 (s, 3H, CH₃).

Step 3: 5-Fluoroisatin (8.2 g, 49.7 mmol) was dissolved in 50 mL of hydrazine hydrate and refluxed for 1 hour. The reaction mixtures were then poured in ice water. The precipitate was then filtered, washed with water and dried under vacuum oven to give 5-fluoro-2-oxindole (7.5 g).

Step 4: The reaction mixture of 5-fluorooxindole (100 mg, 0.66 mmol), 5-formyl-2,4-dimethyl-1H-pyrrole-3-carboxylic acid (133 mg, 0.79 mmol), and 10 drops of piperidine in ethanol (3 mL) was stirred at 60° C. overnight and filtered. The solid was washed with 1 M of aqueous hydrochloride solution, water, and dried to afford 5-(5-fluoro-2-oxo-1,2-dihydro-indol-3-ylidenemethyl)-2,4-dimethyl-1H-pyrrole-3-carboxylic acid (201 mg, quantitative) as a yellow solid. MS m/z (relative intensity, %) 299 ([M−1]⁺, 100).

### Example 4

### Synthesis of 5-(5-Fluoro-2-oxo-1,2-dihydro-indol-3-ylidene-methyl)-2,4-dimethyl-1H-pyrrole-3-carboxylic acid(3-diethylamino-2-hydroxy-propyl)-amide

Step 1: To 2-chloromethyloxirane (95 g, 1.03 mole) was added a mixture of water (3.08 g, 0.17 mole) and diethylamine (106.2 mL, 1.03 mole) at 30° C. The reaction mixture was then stirred at 28-35° C. for 6 hour and cooled to 20-25° C. to give 1-chloro-3-diethylamino-propan-2-ol.

Step 2: A solution of sodium hydroxide (47.9 g, 1.2 mole) in 78 mL water was added 1-chloro-3-diethylamino-propan-2-ol. The resultant was stirred at 20-25° C. for 1 hour, diluted with 178 mL of water and extracted with ether twice. The combined ether solution was dried with solid potassium hydroxide and evaporated to give 135 g of crude product which was purified by fraction distillation to give pure glycidyldiethylarnine (98 g, 76%) as an oil.

Step 3: To the ice-cold solution of ammonium hydroxide (25 mL, 159 mmole) of 25% (w/w) was added glycidyldiethylamine dropwise (3.2 g, 24.8 mmol) over 10 minutes. The reaction mixture was stirred at 0-5° C. for 1 hour and then room temperature for 14 hours. The resulting reaction mixture was evaporated and distilled (84-90° C. at 500-600 mT) to yield 1-amino-3-diethylamino-propan-2-ol (3.3 g, 92%). MS m/z 147 ([M+1]⁺).

Step 4: To the solution of 5-formyl-2,4-dimethyl-1H-pyrrole-3-carboxylic acid (100 mg, 0.43 mmol), EDC (122.7 mg, 0.64 mmol) and HOBt (86.5 mg, 0.64 mmol) in 1.0 mL of DMF was added 1-amino-3-diethylamino-propan-2-ol (93.2 mg, 0.64 mmol). The resulting reaction solution was stirred at room temperature overnight and evaporated. The residue was suspended in 10 mL of water and filtered. The solid was washed with saturated sodium bicarbonate and water and dried in a high vacuum oven overnight to give crude product which was purified on column chromatography eluting with 6% methanol-dichlormethane containing triethylnamine (2 drops/100 mL of 6% methanol-dichloromethane) to give 5-(5-fluoro-2-oxo-1,2-dihydro-indol-3-ylidenemethyl)-2,4-dimethyl-1H-pyrrole-3-carboxylic acid(3-diethylamino-2-hydroxy-propyl)-amide (62 mg, 34%) as a yellow solid.

¹H NMR (400 MHz, DMSO-d6) δ 13.70 (s, 1H, NH-1′), 10.90 (s, 1H, NH-1), 7.76 (dd, J=2.38, 9.33 Hz, 1H, H-4), 7.72 (s, 1H, vinyl-H), 7.60 (m, br., 1H, CONHCH₂CH(OH)—CH₂N(C₂H₅)₂-4′), 6.93 (dt, J=2.38, 8.99 Hz, 1H, H-5), 6.85 (dd, J=4.55, 8.99 Hz, 1H, H-6), 3.83 (m, br, 1H, OH), 3.33 (m, 4H), 2.67 (m, br, 5H), 2.46 (s, 3H, CH₃), 2.44 (s, 3H, CH₃), 1.04 (m, br, 6H, CH₃×2). MS m/z (relative intensity, %) 427 ([M+1]⁺, 100).

### Example 5

### Synthesis of 5-[5-Fluoro-2-oxo-1,2-dihydro-indol-(3Z)-ylidene-methyl]-2,4-dimethyl-1H-pyrrole-3-carboxylic acid(2-hydroxy-3-morpholin-4-yl-propyl)-amide (R), (S) and (R/S) (Compounds 4, 5 and 6)

Step 1: A mixture of morpholine (2.6 mL, 30 mmol) and epichlorohydrin (2.35 mL, 30 mmol) in ethanol (50 mL) was stirred at 70° C. overnight. After removing the solvent, the residue was diluted with methylene chloride (50 mL). The clear solid precipitated was collected by vacuum filtration to give 1-chloro-3-morpholin-4-yl-propan-2-ol (2.0 g, 37%). ¹H NMR (DMSO-d₆) δ 3.49 (t, J=4.8 Hz, 2H), 3.60 (t, J=4.6 Hz, 2H), 3.75 (m, 4H, 2×CH₂), 4.20 (dd, J=5.2, 12 Hz, 2H), 4.54 (m, 2H), 4.62 (m, 1H, CH), 6.64 (d, J=6.4 Hz, 1H, OH). MS (m/z) 180.2 (M+1).

Step 2: 1-Chloro-3-morpholin-4-yl-propan-2-ol (2.0 g, 11 mmol) was treated with the solution of NH₃ in methanol (25% by weight, 20 mL) at room temperature. Nitrogen was bubbled into the reaction mixture to remove the ammonia. Evaporation of solvent gave the hydrogen chloride salt of 1-amino-3-morpholin-4-yl-propan-2-ol (2.0 g, 91%). ¹H NMR (DMSO-d₆) δ 2.30 (d, J=6.0 Hz, 2H), 2.36 (m, 4H, NCH₂), 2.65 (dd, J=8.4, 12.8 Hz, 1H), 2.91 (dd, J=3.6, 12.8 Hz, 1H), 3.52 (m, 4H, OCH₂), 3.87 (m, 1H, CH), 5.32 (s, 1H, OH), 8.02 (brs., 3H, NH₃⁺). MS (m/z) 161.1 (M+1).

Step 3: 5-(5-Fluoro-2-oxo-1,2-dihydro-indol-3-ylidenemethyl)-2,4-dimethyl-1H-pyrrole-3-carboxylic acid (120 mg, 0.4 mmol) was condensed with 1-amino-3-morpholin-4-yl-propan-2-ol(74 mg, 0.48 mmol) to precipitate 5-[5-fluoro-2-oxo-1,2-dihydro-indol-(3Z)-ylidenemethyl]-2,4-dimethyl-1H-pyrrole-3-carboxylic acid(2-hydroxy-3-morpholin-4-yl-propyl)-amide (65 mg, 36%). The mother liquid was evaporated to dryness and the residue was purified by flash chromatography to give additional 2N (70 mg, 39%). ¹H NMR (DMSO-d₆) δ 2.28 (m, 1H), 2.32 (m, 1H), 2.40 (m, 4H), 2.40, 2.42 (2×s, 6H, 2×CH₃), 3.15 (s, 1H), 3.31 (m, 1H), 3.55 (m, 4H), 3.78 (m, 1H), 4.73 (brs, 1H, OH), 6.82 (dd, J=4.5, 8.4 Hz, 1H), 6.90 (td, ²J=2.8, ³J=10.0 Hz, 1H), 7.53 (m, 1H), 7.70 (s, 1H), 7.74 (dd, J=2.0, 9.6 Hz, 1H) (aromatic and vinyl), 10.87 (s, 1H, CONH), 13.66 (s, 1H, NH). LC-MS (m/z) 441.4 (M−1).

### SYNTHESIS OF 2-HYDROXY-7-OXA-4-AZONIASPIRO[3.5]NONANE CHLORIDE

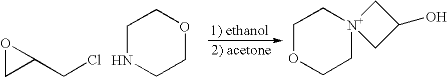

To a 1 L 3-neck round bottom flask, fitted with a thermocouple, nitrogen inlet and a 250 mL addition funnel, was charged morpholine (91.5 g, 91.5 mL, 1.05 mole, 1.0 eq.) and 100 mL of ethanol. The solution was stirred rapidly while adding epichlorohydrin (10 g, 84.5 mL, 1.08 mole, 1.03 eq.) from the addition funnel over about 30 minutes. The temperature was monitored and when the pot temperature reached 27° C., the reaction was cooled with an ice water bath. The clear solution was stirred for 18 hours. The reaction was assayed by GC (dilute 5 drops of reaction mixture into 1 mL of ethanol and inject onto a 15 m DB-5 capillary GC column with the following run parameters, Injector 250° C., detector 250° C., initial oven temperature 28° C. warming to 250° C. at 10° C. per minute.) The reaction was complete with less than 3% morpholine remaining. The reaction was concentrated on the rotoevaporated at 50° C. with full house vacuum until no more distillate could be condensed. The resulting oil was stored at room temperature for 24-48 hours or until a significant mass of crystals was observed (seeded will speed up the process). The slurry was diluted with 250 mL of acetone and filtered. The solids were dried in the vacuum oven at 60° C. for 18-24 hours. This provided 84 g of crystalline product. The mother liquors could be concentrated and the crystallization process repeated in increase recovery. ¹H NMR (400 MHz, DMSO-d₆) δ 6.55 (d, 1 H), 4.64 (m, 1 H), 4.53 (m, 2 H), 4.18 (m, 2 H), 3.74 (m, 4 H), 3.60 (m, 2 H), 3.48 (m, 2 H). ¹³C NMR (100 MHz, DMSO-d₆) δ 70.9, 61.39, 61.04, 60.25, 58.54, 57.80.

### SYNTHESIS OF 1-AMINO-3-(4-MORPHOLINYL)-2-PROPANOL (RACEMIC)

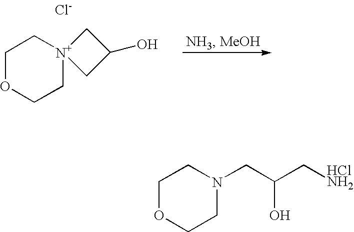

To a 3 L 1-neck round bottom flask with a magnetic stir bas was charged 2-hydroxy-7-oxa-4-azoniaspiro[3.5]nonane chloride (150 g, 835 mmole) followed by 23 wt. % anhydrous ammonia in methanol (2120 mL). The flask was stoppered and the resulting clear solution was stirred at 20-23° C. for 18 hours. GC under the conditions above showed no remaining starting material. The stopper was removed and the ammonia allowed to bubble out of the solution for 30 minutes. The flask was then transferred to a rotoevaporated and concentrated to a white solid with 45° C. bath and full house vacuum. ¹H NMR (400 MHz, DMSO-d₆) δ 3.57 (dd, 2H), 3.3-3.5 (m, 6 H), 2.59 (m, 2 H), 2.2-2.4 (m, 6 H); ¹³C NMR (100 MHz DMSO-d₆) δ 70.8, 67.1, 60.1, 53.8, 48.1.

Following the procedure described in Example 3 but substituting 2-(RS)-1-amino-3-morpholin-4-yl-propan-2-ol with 2-(S)-1-amino-3-morpholin-4-yl-propan-2-ol prepared as described below the desired compound 5-[5-fluoro-2-oxo-1,2-dihydro-indol-(3Z)-ylidenemethyl]-2,4-dimethyl-1H-pyrrole-3-carboxylic acid(2-(S)-hydroxy-3-morpholin-4-yl-propyl)-amide was obtained.

### SYNTHESIS OF 1-AMINO-3-(4-MORPHOLINYL)-2-PROPANOL (NON-RACEMIC)

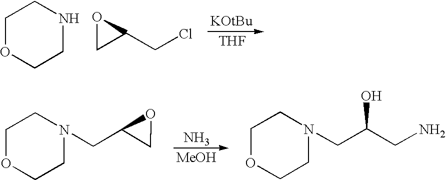

To 1 L 3-neck round bottom flask, fitted with mechanical stirring, thermocouple and addition funnel, was charged morpholine (91.5 g, 91.5 mL, 1.05 mole, 1.0 eq.) and 45 mL of t-butanol. The solution was stirred rapidly while adding R-epichlorohydrin (10 g, 84.5 mL, 1.08 mole. 1.03 eq.) from the addition funnel over about 30 minutes. The temperature was monitored and when the pot temperature reached 27° C., the reaction was cooled with an ice water bath. The clear solution was stirred for 18 hours. The reaction was assayed by GC (dilute 5 drops of reaction mixture into 1 mL of ethanol and inject onto a 15 m DB-5 capillary GC column with the following run parameters, Injector 250° C., detector 250° C., initial oven temperature 28° C. warming to 250° C. at 10° C. per minute). The reaction was complete with less than 3% morpholine remaining. The solution was cooled to 10° C. and a 20 wt % solution of potassium t-butoxide in THF (576 g) was added dropwise keeping the temperature less than 15° C. The resulting white slurry was stirred at 10-15° C. for 2 hours and checked by GC using the above conditions. None of the chlorohydrin could be observed. The mixture was concentrated on the rotoevaporated using 50° C. bath and full house vacuum. The resulting mixture was diluted with water (500 mL) and methylene chloride. The phases were separated and the aqueous phase washed with methylene chloride (500 mL). The combined organic layers were dried over sodium sulfate and concentrated to a clear, colorless oil. This provided 145 g, 97% yield of the epoxide. ¹H NMR (400 MHz, DMSO-d₆) δ 3.3 (dd, 4 H), 3.1 (m, 1 H), 2.6 (dd, 1 H), 2.5 (dd, 1 H), 2.4 (m, 4 H), 2.2 (dd, 2 H); ¹³C NMR (100 MH_{z}, DMSO-d₆) δ 65.4, 60.1, 53.1, 48.9, 43.4.

The above crude epoxide was charged to a 3 L 1-neck round bottom flask with a magnetic stir bar. Anhydrous ammonia in methanol (24% w/w 2.5 L) was added, the flask was stoppered and the mixture stirred at room temperature for 24 hours. GC under the conditions above showed no remaining starting material. The stopper was removed and the ammonia allowed to bubble out of the solution for 30 minutes. The flask was then transferred to a rotoevaporated and concentrated to a clear colorless oil with 45° C. bath and full house vacuum. This provided 124 g of product. ¹H NMR (400 MH_{z}, DMSO-d₆) δ 3.57 (dd, 2H), 3.3-3.5 (m, 6 H), 2.59 (m, 2 H), 2.2-2.4 (m, 6 H); ¹³C NMR (100 MH_{z}, DMSO-d₆) δ 70.8, 67.1, 60.1, 53.8, 48.1.

### SYNTHESIS OF 1-AMINO-3-(4-MORPHOLINYL)-2-(S)-PROPANOL

To 1 L 3-neck round bottom flask, fitted with mechanical stirring, thermocouple and addition funnel, was charged morpholine (91.5 g, 91.5 mL, 1.05 mole, 1.0 eq.) and 200 mL of methanol. The solution was stirred rapidly while adding R-epichlorohydrin (10 g, 84.5 mL, 1.08 mole, 1.03 eq.) from the addition funnel over about 30 minutes. The temperature was monitored and when the pot temperature reached 27° C., the reaction was cooled with an ice water bath. The clear solution was stirred for 18 hours. The reaction was assayed by GC (dilute 5 drops of reaction mixture into 1 mL of ethanol and inject onto a 15 m DB-5 capillary GC column with the following run parameters, Injector 250° C., detector 250° C., initial oven temperature 28° C. warming to 250° C. at 10° C. per minute.) The reaction was complete with less than 3% morpholine remaining. The solution was cooled to 10° C. and a 25 wt. % solution of sodium methoxide in methanol (233 g, 1.08 mole, 247 mL) was added dropwise keeping the temperature less than 15° C. The resulting white slurry was stirred at 10-15° C. for 2 hours and checked by GC using the above conditions. None of the chlorohydrin could be observed. The mixture was concentrated on the rotoevaporator using 50° C. bath and full house vacuum. The resulting mixture was diluted with water (500 mL) and methylene chloride. The phases were separated and the aqueous phase washed with methylene chloride (500 mL). The combined organic layers were dried over sodium sulfate and concentrated to a clear, colorless oil. This provided 145 g, 97% yield of 1,2-epoxy-3-morpholin-4-ylpropane. ¹H NMR (400 MHz, DMSO-d₆) δ 3.3 (dd, 4 H), 3.1 (m, 1 H), 2.6 (dd, 1 H), 2.5 (dd, 1 H), 2.4 (m, 4 H), 2.2 (dd, 2 H); ¹³C NMR (100 MHz, DMSO-d₆) δ 65.4, 60.1, 53.1, 48.9, 43.4.

The above crude 1,2-epoxy-3-morpholin-4-ylpropane was charged to a 3 L 1-neck round bottom flask with a magnetic stir bar. Anhydrous ammonia in methanol (24% w/w 2.5 L) was added, the flask was stoppered and the mixture stirred at room temperature for 24 hours. GC under the conditions above showed no remaining starting material. The stopper was removed and the ammonia allowed to bubble out of the solution for 30 minutes. The flask was then transferred to a rotoevaporated and concentrated to a clear colorless oil with 45° C. bath and full house vacuum. This provided 124 g of 1-amino-3-(4-morpholinyl)-2-(S)-propanol.

¹H NMR (400 MHz, DMSO-d₆) δ 3.57 (dd,2H), 3.3-3.5 (m, 6 H), 2.59 (m, 2 H), 2.2-2.4 (m, 6 H); ¹³C NMR (100 MHz, DMSO-d₆) δ 70.8, 67.1, 60.1, 53.8, 48.1.

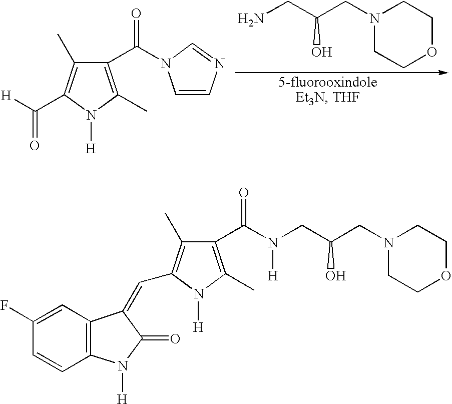

Imidazole amide (7.0 g, 32.3 mmol), amine (15.0 g, 64.6 mmol), 5-fluorooxindole (4.93 g, 32.6 mmol), triethylamine (9.79 g, 96.9 mmol), and THF (88 mL) were mixed and heated to 60° C. A brown solution formed. After stirring for 24 h at 60° C., the yellow slurry was cooled to rt (room temperature) and filtered. The cake was washed with 80 mL THF and dried overnight at 50° C. under house vacuum. A brown solid (23.2 g) was obtained. The solid was slurried in 350 mL water for 5 h at rt and filtered. The cake was washed with 100 mL water and dried at 50° C. under house vacuum overnight. 8.31 g were obtained with 56% chemical yield.

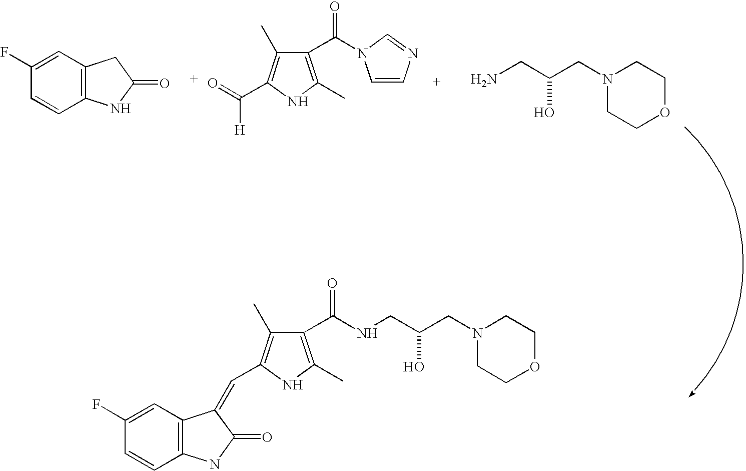

A 0.25 L flask fitted with a thermometer, condenser, magnetic stirring, and nitrogen inlet was charged with 4.92 g 5-Fluorooxindole, 7.0 g Imidazole amide, 15.5 g (R)-1-Amino-3-(4-morpholinyl)-2-propanol, 9.78 g Triethylamine and 88 mL Tetrahydrofuran. The mixture was heated to 60° C. for 16.5 hours. The reaction is cooled to ambient temperature and filtered. The solids obtained are slurried (3) three successive times in acetonitrile at 11 mL/g, dried in vacuo for 3.6 g (25.25%). [HPLC, Hypersil BDS, C-18, 5μ, (6:4), Acetonitrile: 0.1M Ammonium Chloride, PHA-571437=4.05 min.] H¹NMR (DMSO): δ 10.86 (1H,bs); 7.75 (1H,d); 7.70 (1H,s); 7.50 (1H,m); 6.88 (2H,m); 4.72 (1H,bs); 3.78 (1H,bs); 3.56 (4H,m); 3.32 (6H, m); 3.15 (1H,m); 2.43 (8H,bm).

### Example 6

### Synthesis of 2,4-dimethyl-5-[2-oxo-1,2-dihydro-indol-(3Z)-ylidenemethyl]-1H-pyrrole-3-carboxylic acid(2-hydroxy-3-morpholin-4-yl-propyl)-amide

5-(2-Oxo-1,2-dihydro-indol-3-ylidenemethyl)-2,4-dimethyl-1H-pyrrole-3-carboxylic acid (113 mg, 0.4 mmol) was condensed with 1-amino-3-morpholin-4-yl-propan-2-ol (74 mg, 0.48 mmol) to precipitate 2,4-dimethyl-5-[2-oxo-1,2-dihydro-indol-(3Z)-ylidenemethyl]-1H-pyrrole-3-carboxylic acid(2-hydroxy-3-morpholin-4-yl-propyl)-amide (77 mg, 45.3%).

¹H NMR (DMSO-d₆) δ 2.27 (m, 1H), 2.32 (m, 1H), 2.40 (m, 4H), 2.40, 2.42 (2×s, 6H, 2×CH₃), 3.15 (s, 1H), 3.32 (m, 1H), 3.55 (m, 4H), 3.77 (m, 1H), 4.74 (d, J=4.8 Hz, 1H, OH), 6.86 (d, J=7.6 Hz, 1H), 6.96 (t, J=7.2 Hz, 1H), 7.10 (t, J=7.6 Hz, 1H), 7.49 (t, J=5.6 Hz, 1H), 7.61 (s, 1H), 7.77 (d, J=8.0 Hz, 1H) (aromatic and vinyl), 10.88 (s, 1H, CONH), 13.62 (s, 1H, NH). LC-MS (m/z) 425.4 (M+1).

### Example 7

### Synthesis of 5-[5-chloro-2-oxo-1,2-dihydro-indol-(3Z)-ylidene-methyl]-2,4-dimethyl-1H-pyrrole-3-carboxylic acid(2-hydroxy-3-morpholin-4-yl-propyl)-amide (Compound 7)

5-(5-Chloro-2-oxo-1,2-dihydro-indol-3-ylidenemethyl)-2,4-dimethyl-1H-pyrrole-3-carboxylic acid (126.6 mg, 0.4 mmol) was condensed with 1-amino-3-morpholin-4-yl-propan-2-ol (74 mg, 0.48 mmol) to precipitate 5-[5-Chloro-2-oxo-1,2-dihydro-indol-(3Z)-ylidenemethyl]-2,4-dimethyl-1H-pyrrole-3-carboxylic acid(2-hydroxy-3-morpholin-4-yl-propyl)-amide (107 mg, 58%).

¹H NMR (DMSO-d₆) δ 2.29 (m, 1H), 2.33 (m, 1H), 2.39(m, 4H), 2.40, 2.42 (2×s, 6H, 2×CH₃), 3.15 (s, 1H), 3.37 (m, 1H), 3.55 (m, 4H), 3.77 (m, 1H), 4.74 (d, J=4.8 Hz, 1H, OH), 6.85 (d, J=8.4 Hz, 1H), 7.11 (dd, J=2.0, 8.0 Hz, 1H), 7.53 (t, J=5.6 Hz, 1H), 7.75 (s, 1H), 7.97 (d, J=2.0 Hz, 1H) (aromatic and vinyl), 10.99 (s, 1H), CONH), 13.62 (s, 1H, NH). LC-MS (m/z) 457.4 (M-−1).

### Example 8

### -Synthesis of 5-[5-bromo-2-oxo-1,2-dihydro-indol-(3Z)-ylidene-methyl]-2,4-dimethyl-1H-pyrrole-3-carboxylic acid(2-hydroxy-3-morpholin-4-yl-propyl)-amide

5-(5-Bromo-2-oxo-1,2-dihydro-indol-3-ylidenemethyl)-2,4-dimethyl-1H-pyrrole-3-carboxylic acid (72.2 mg, 0.2 mmol) was condensed with 1-amino-3-morpholin-4-yl-propan-2-ol (38 mg, 0.24 mmol) to precipitate 5-[5-Bromo-2-oxo-1,2-dihydro-indol-(3Z)-ylidenemethyl]-2,4-dimethyl-1H-pyrrole-3-carboxylic acid(2-hydroxy-3-morpholin-4-yl-propyl)-amide (55 mg, 55%).

¹H NMR (DMSO-d₆) δ 2.27 (m, 1H), 2.32 (m, 1H), 2.39(m, 4H), 2.41, 2.42 (2×s, 6H, 2×CH₃), 3.13 (s, 1H), 3.35 (m, 1H), 3.55 (m, 4H), 3.77 (m, 1H), 4.74 (d, J=4.4 Hz, 1H, OH), 6.80 (d, J=8.4 Hz, 1H), 7.24 (dd, J=2.0, 8.0 Hz, 1H), 7.51 (t, J=5.6 Hz, 1H), 7.76 (s, 1H), 8.09 (d, J=2.0 Hz, 1H) (aromatic and vinyl), 10.99 (s, 1H), CONH), 13.62 (s, 1H, NH). LC-MS (m/z) 503.4 (M−1).

### Example 9

### —Synthesis of 2,4-dimethyl-5-[2-oxo-1,2-dihydro-indol-(3Z)-ylidene-methyl]-1H-pyrrole-3-carboxylic acid(2-hydroxy-3-[1,2,3]triazol-1-yl-propyl)-amide

Step 1: A mixture of 3-[1,2,3]triazole (2.0 g, 29 mmol), epichlorohydrin (3.4 mL, 43.5 mmol) and N,N-diisopropyl-ethylamine (2.6 mL, 15 mmol) in ethanol (50 mL) was stirred at room temperature overnight. After removing the solvents, the residue was purified by flash chromatography (CH₂Cl₂/CH₃OH=100/1-100/2-100/4) to give 1-chloro-3-(1,2,3)-triazol-2-ylpropan-2-ol (2.1 g, 45%). ¹H NMR (CDCl₃) δ 3.52 (m, 2H, OH and CH₂), 3.60 (dd, J=5.2, 11.2 Hz, 1H), 4.36 (m, 1H, CH), 4.68 (m, 2H), 7.67 (s, 2H). MS (m/z) 162.1 (M+1) and 1-chloro-3-(1,2,3)triazol-1-ylpropan-2-ol (2.3 g, 49%). ¹H NMR (CDCl₃) δ 3.56 (s, 1H), 3.57 (s, 1H), 4.35 (m, 1H), 4.53 (dd, J=7.2, 14 Hz, 1H), 4.67 (dd, J=3.8, 14 Hz, 1H), 7.67 (s, 1H), 7.71 (s, 1H). MS (m/z) 162.1 (M+1).

Step 2: 1-Chloro-3(1,2,3)triazol-1-ylpropan-2-ol (2.3 g, 13 mmol) was treated with the solution of NH₃ in methanol (25% by weight, 20 mL) at 60° C. overnight in a sealed pressure vessel. After cooling to room temperature, nitrogen was bulbbed into the reaction mixture to remove the ammonia. Evaporation of solvent gave the hydrogen chloride salt of 1-amino-3-(1,2,3)triazol-1-ylpropan-2-ol (2.57 g, 100%).

¹H NMR (DMSO-d₆) δ 2.68 (dd, J=8.8, 12.8 Hz, 1H), 2.97 (dd, J=3.6, 12.8 Hz, 1H), 4.15 (m, 1H), 4.44 (dd, J=6.4, 14 Hz, 1H), 4.57 (dd, J=4.6, 14 Hz, 1H), 5.95 (d, J=5.2 Hz, 1H, OH), 7.77 (s, 1H), 8.01 (brs., 3H, NH₃⁺), 8.12 (s, 1H). MS (m/z) 143.1 (M+1).

Step 3: 5-(2-Oxo-1,2-dihydro-indol-3-ylidenemethyl)-2,4-dimethyl-1H-pyrrole-3-carboxylic acid (113 mg, 0.4 mmol) was condensed with 1-amino-3(1,2,3)triazole-1-yl-propan-2-ol (85 mg, 0.48 mmol) to precipitate 2,4-dimethyl-5-[2-oxo-1,2-dihydro-indol-(3Z)-ylidenemethyl]-1H-pyrrole-3-carboxylic acid(2-hydroxy-3-[1,2,3]triazol-1-yl-propyl)-amide (70 mg, 41%).

¹H NMR (DMSO-d₆) δ 2.45, 2.48 (2×s, 6H, 2×CH₃), 3.35 (m, 2H), 4.02 (m, 1H), 4.32 (dd, J=7.6, 14 Hz,1H), 4.53 (dd, J=3.4, 14 Hz,1H), 5.43 (d, J=5.6 Hz, 1H, OH), 6.91 (d, J=7.6 Hz, 1H), 7.01 (t, J=7.6 Hz, 1H), 7.15 (t, J=8.0 Hz, 1H), 7.66 (s, 1H), 7.12 (t, J=5.6 Hz, 1H), 7.74 (s, 1H), 7.77 (d, J=7.6 Hz, 1H), 8.11 (s, 1H), 10.93 (s, 1H, CONH), 13.68 (s, 1H, NH). LC-MS (m/z) 405.4 (M−1).

### Example 10

### Synthesis of 5-[5-fluoro-2-oxo-1,2-dihydro-indol-(3Z)-ylidene-methyl]-2,4-dimethyl-1H-pyrrole-3-carboxylic acid(2-hydroxy-3-[1,2,3]triazol-1-yl-propyl)-amide

5-(5-Fluoro-2-oxo-1,2-dihydro-indol-3-ylidenemethyl)-2,4-dimethyl-1H-pyrrole-3-carboxylic acid (120 mg, 0.4 mmol) was condensed with 1-amino-3(1,2,3)triazol-1-yl-propan-2-ol (85 mg, 0.48 mmol) to precipitate 5-[5-fluoro-2-oxo-1,2-dihydro-indol-(3Z)-ylidenemethyl]-2,4-dimethyl-1H-pyrrole-3-carboxylic acid(2-hydroxy-3-[1,2,3]triazol-1-yl-propyl)-amide (100 mg, 62%).

¹H NMR (DMSO-d₆) δ 2.42, 2.44 (2×s, 6H, 2×CH₃), 3.27 (m, 2H), 3.98 (m, 1H), 4.27 (dd, J=7.6, 14 Hz,1H), 4.50 (dd, J=3.4, 13.6 Hz,1H), 5.38 (d, J=5.6 Hz, 1H, OH), 6.82 (dd, J=4.4, 8.4 Hz, 1H), 6.91 (td, ²J=2.4, ³J=9.0 Hz, 1H), 7.70 (m, 3H), 7.75 (dd, J=2.4, 9.2 Hz, 1H), 8.11 (s. 1H), 10.93 (s, 1H, CONH), 13.73 (s, 1H, NH). LC-MS (m/z) 423.4 (M−1).

### Example 11

### Synthesis of 5-[5-chloro-2-oxo-1,2-dihydro-indol-(3Z)-ylidene-methyl]-2,4-dimethyl-1H-pyrrole-3-carboxylic acid(2-hydroxy-3-[1,2,3]triazol-1-yl-propyl)-amide

5-(5-Chloro-2-oxo-1,2-dihydro-indol-3-ylidenemethyl)-2,4-dimethyl-1H-pyrrole-3-carboxylic acid (126.6 mg, 0.4 mmol) was condensed with 1-amino-3(1,2,3)triazole-1-yl-propan-2-ol (85 mg, 0.48 mmol) to precipitate 5-[5-Chloro-2-oxo-1,2-dihydro-indol-(3Z)-ylidenemethyl]-2,4-dimethyl-1H-pyrrole-3-carboxylic acid(2-hydroxy-3-[1,2,3]triazol-1-yl-propyl)-amide (48 mg, 27%).

¹H NMR (DMSO-d₆) δ 2.42, 2.44 (2×s, 6H, 2×CH₃), 3.27 (m, 2H), 3.99 (m, 1H), 4.28 (dd, J=7.8, 14 Hz,1H), 4.51 (dd, J=3.2, 14 Hz,1H), 5.39 (d, J=6.0 Hz, 1H, OH), 6.85 (d, J=8.4 Hz, 1H), 7.12 (dd, J=2.0, 8.2 Hz, 1H), 7.70 (m, 2H), 7.74 (s, 1H), 7.97 (d, J=2.0 Hz, 1H), 8.07 (s, 1H), 10.99 (s, 1H, CONH), 13.65 (s, 1H, NH). LC-MS (m/z) 439.4 (M−1).

### Example 12

### Synthesis of 5-[5-bromo-2-oxo-1,2-dihydro-indol-(3Z)-ylidene-methyl]-2,4-dimethyl-1H-pyrrole-3-carboxylic acid(2-hydroxy-3-[1,2,3]triazol-1-yl-propyl)-amide

5-(5-Bromo-2-oxo-1,2-dihydro-indol-3-ylidenemethyl)-2,4-dimethyl-1H-pyrrole-3-carboxylic acid (144.4 mg, 0.4 mmol) was condensed with 1-amino-3(1,2,3)triazole-1-yl-propan-2-ol (85 mg, 0.48 mmol) to precipitate 5-[5-bromo-2-oxo-1,2-dihydro-indol-(3Z)-ylidenemethyl]-2,4-dimethyl-1H-pyrrole-3-carboxylic acid(2-hydroxy-3-[1,2,3]triazol-1-yl-propyl)-amide (130 mg, 67%).

¹H NMR (DMSO-d₆) δ 2.41, 2.44 (2×s, 6H, 2×CH₃), 3.27 (m, 2H), 3.99 (m, 1H), 4.28 (dd, J=7.6, 14 Hz,1H), 4.50 (dd, J=3.6, 14 Hz,1H), 5.40 (d, J=5.6 Hz, 1H, OH), 6.81 (d, J=8.4 Hz, 1H), 7.24 (dd, J=2.0, 8.0 Hz, 1H), 7.70 (m, 2H), 7.77 (s, 1H), 8.07 (s. 1H), 8.10 (d, J=1.6 Hz, 1H), 11.0 (s, 1H, CONH), 13.64 (s, 1H, NH). LC-MS (m/z) 485.4 (M−1).

### Example 13

### 5-(5-Fluoro-2-oxo-1,2-dihydroindol-3-ylidenemethyl)-2,4-dimethyl-1H-pyrrole-3-carboxylic acid(2-diethylamino-ethyl)amide (Compound 1)

5-Fluoro-1,3-dihydroindol-2-one (0.54 g, 3.8 mmol) was condensed with 5-formyl-2,4-dimethyl-1H-pyrrole-3-carboxylic acid(2-diethylaminoethyl)amide to give 0.83 g (55%) of the title compound as a yellow green solid.

¹HNMR (360 MHz, DMSO-d₆) δ 13.66 (s, 1H, NH), 10.83 (s, br, 1H, NH), 7.73 (dd, J=2.5 & 9.4 Hz, 1H), 7.69 (s, 1H, H-vinyl), 7.37 (t, 1H, CONHCH₂CH₂), 6.91 (m, 1H), 6.81-6.85 (m, 1H), 3.27 (m, 2H, CH₂), 2.51 (m, 6H, 3×CH₂), 2.43 (s, 3H, CH₃), 2.41 (s, 3H, CH₃), 0.96 (t, J=6.9 Hz, 6H, N(CH₂CH₃)₂). MS-EI m/z 398 [M+].

### Alternative synthesis of 5-(5-Fluoro-2-oxo-1,2-dihydroindol-3-ylidenemethyl)-2,4-dimethyl-1H-pyrrole-3-carboxylic acid(2-diethylamino-ethyl)amide

Hydrazine hydrate (55%, 3000 mL) and 5-fluoroisatin (300 g) were heated to 100° C. An additional 5-fluoro-isatin (500 g) was added in portions (100 g) over 120 minutes with stirring. The mixture was heated to 110° C. and stirred for 4 hours. The mixture was cooled to room temperature and the solids collected by vacuum filtration to give crude (2-amino-5-fluoro-phenyl)-acetic acid hydrazide (748 g). The hydrazide was suspended in water (700 mL) and the pH of the mixture adjusted to <pH 3 with 12 N hydrochloric acid. The mixture was stirred for 12 hours at room temperature. The solids were collected by vacuum filtration and washed twice with water. The product was dried under vacuum to give 5-fluoro-1,3-dihydro-indol-2-one (600 g, 73% yield) as a brown powder. ¹H-NMR (dimethylsulfoxide-d₆) δ 3.46 (s, 2H, CH₂), 6.75, 6.95, 7.05 (3× m, 3H, aromatic), 10.35 (s, 1H, NH). MS m/z 152 [M+1].

3,5-Dimethyl-1H-pyrrole-2,4-dicarboxylic acid 2-tert-butyl ester 4-ethyl ester (2600 g) and ethanol (7800 mL) were stirred vigorously while 10 N hydrochloric acid (3650 mL) was slowly added. The temperature increased from 25° C. to 35° C. and gas evolution began. The mixture was warmed to 54° C. and stirred with further heating for one hour at which time the temperature was 67° C. The mixture was cooled to 5° C. and 32 L of ice and water were slowly added with stirring. The solid was collected by vacuum filtration and washed three times with water. The solid was air dried to constant weight to give of 2,4-dimethyl-1H-pyrrole-3-carboxylic acid ethyl ester (1418 g, 87% yield) as a pinkish solid. ¹H-NMR (dimethylsulfoxide-d₆) δ 2.10, 2.35 (2×s, 2×3H, 2×CH₃), 4.13 (q, 2H, CH₂), 6.37 (s, 1H, CH), 10.85 (s, 1H, NH). MS m/z 167 [M+1].

Dimethylformamide (322 g) and dichloromethane (3700 mL) were cooled in an ice bath to 4° C. and phosphorus oxychloride (684 g) was added with stirring. Solid 2,4-dimethyl-1H-pyrrole-3-carboxylic acid ethyl ester (670 g) was slowly added in aliquots over 15 minutes. The maximum temperature reached was 18° C. The mixture was heated to reflux for one hour, cooled to 10° C. in an ice bath and 1.6 L of ice water was rapidly added with vigorous stirring. The temperature increased to 15° C. 10 N Hydrochloric acid (1.6 L) was added with vigorous stirring. The temperature increased to 22° C. The mixture was allowed to stand for 30 minutes and the layers allowed to separate. The temperature reached a maximum of 40° C. The aqueous layer was adjusted to pH 12-13 with 10 N potassium hydroxide (3.8 L) at a rate that allowed the temperature to reach and remain at 55° C. during the addition. After the addition was complete the mixture was cooled to 10° C. and stirred for 1 hour. The solid was collected by vacuum filtration and washed four times with water to give 5-formyl-2,4-dimethyl-1H-pyrrole-3-carboxylic acid ethyl ester (778 g, 100% yield) as a yellow solid. ¹H-NMR (DMSO-d₆) δ 1.25 (t, 3H, CH₃), 2.44, 2.48 (2×s, 2×3H, 2×CH₃), 4.16 (q, 2H, CH₂), 9.59 (s, 1H, CHO), 12.15 (br s, 1H, NH). MS m/z 195 [M+1].

5-Formyl-2,4-dimethyl-1H-pyrrole-3-carboxylic acid ethyl ester (806 g), potassium hydroxide (548 g), water (2400 mL ) and methanol (300 mL) were refluxed for two hours with stirring and then cooled to 8° C. The mixture was extracted twice with dichloromethane. The aqueous layer was adjusted to pH 4 with 1000 mL of 10 N hydrochloric acid keeping the temperature under 15° C. Water was added to facilitate stirring. The solid was collected by vacuum filtration, washed three times with water and dried under vacuum at 50° C. to give 5-formyl-2,4-dimethyl-1H-pyrrole-3-carboxylic (645 g, 93.5% yield) acid as a yellow solid. ¹H-NMR (DMSO-d₆) δ 2.40, 2.43 (2×s, 2×3H, 2×CH₃), 9.57 (s, 1H, CHO), 12.07 (br s, 2H, NH+COOH). MS m/z 168 [M+1].

5-Formyl-2,4-dimethyl-1H-pyrrole-3-carboxylic acid (1204 g) and 6020 mL of dimethylformamide were stirred at room temperature while 1-(3-dimethyl-aminopropyl-3-ethylcarbodiimide hydrochloride (2071 g), hydroxybenzotriazole (1460 g), triethylamine (2016 mL) and diethylethylenediamine (1215 mL) were added. The mixture was stirred for 20 hours at room temperature. The mixture was diluted with 3000 mL of water, 2000 mL of brine and 3000 mL of saturated sodium bicarbonate solution and the pH adjusted to greater than 10 with 10 N sodium hydroxide. The mixture was extracted twice with 5000 mL each time of 10% methanol in dichloromethane and the extracts combined, dried over anhydrous magnesium sulfate and rotary evaporated to dryness. The mixture was with diluted with 1950 mL of toluene and rotary evaporated again to dryness. The residue was triturated with 3:1 hexane:diethyl ether (4000 mL). The solids were collected by vacuum filtration, washed twice with 400 mL of ethyl acetate and dried under vacuum at 34° C. for 21 hours to give 5-formyl-2,4-dimethyl-1H-pyrrole-3-carboxylic acid(2-diethylamino-ethyl)-amide (819 g, 43% yield) as a light brown solid. ¹H-NMR (dimethylsulfoxide-d₆) δ 0.96 (t, 6H, 2×CH₃), 2.31, 2.38 (2×s, 2× CH₃), 2.51 (m, 6H 3×CH₂), 3.28 (m, 2H, CH₂ ), 7.34 (m, 1H, amide NH), 9.56 (s, 1H, CHO), 11.86 (s, 1H, pyrrole NH). MS m/z 266 [M+1].

5-Formyl-2,4-dimethyl-1H-pyrrole-3-carboxylic acid(2-diethylaminoethyl)-amide (809 g), 5-fluoro-1,3-dihydro-indol-2-one (438 g), ethanol (8000 mL) and pyrrolidine (13 mL) were heated at 78° C. for 3 hours. The mixture was cooled to room temperature and the solids collected by vacuum filtration and washed with ethanol. The solids were stirred with ethanol (5900 mL) at 72° C. for 30 minutes. The mixture was cooled to room temperature. The solids were collected by vacuum filtration, washed with ethanol and dried under vacuum at 54° C. for 130 hours to give 5-[5-fluoro-2-oxo-1,2-dihydro-indol-(3Z)-ylidenemethyl]-2,4-dimethyl-1H-pyrrole-3-carboxylic acid(2-diethylamino-ethyl)-amide (1013 g, 88% yield) as an orange solid.

¹H-NMR (dimethylsulfoxide-d6) δ 0.98 (t, 6H, 2×CH3), 2.43, 2.44 (2×s, 6H, 2×CH3), 2.50 (m, 6H, 3×CH2), 3.28 (q, 2H, CH2), 6.84, 6.92, 7.42, 7.71, 7.50 (5×m, 5H, aromatic, vinyl, CONH), 10.88 (s, 1H, CONH), 13.68 (s, 1H, pyrrole NH). MS m/z 397 [M−1].

The malic acid salt of 5-(5-Fluoro-2-oxo-1,2-dihydroindol-3-ylidenemethyl)-2,4-dimethyl-1H-pyrrole-3-carboxylic acid(2-diethylamino-ethyl)amide can be prepared according to the disclosure of U.S. patent Publication No. 2003/0069298 and WO 03/016305, the disclosures of which are incorporated by reference in their entireties.

Synthesis of 5-(5-bromo-2-oxo-1,2-dihydro-indol-3-ylidenemethyl)-2,4-dimethyl-1H-pyrrole-3-carboxylic acid, 5-(5-chloro-2-oxo-1,2-dihydro-indol-3-ylidenemethyl)-2,4-dimethyl-1H-pyrrole-3-carboxylic acid and 5-(2-oxo-1,2-dihydro-indol-3-ylidenemethyl)-2,4-dimethyl-1H-pyrrole-3-carboxylic acid is described in U.S. Pat. No. 6,573,293, the disclosure of which is incorporated herein in its entirety.

### Example 14

### 5-(5-Fluoro-2-oxo-1,2-dihydro-indol-3-ylidenemethyl)-2,4-dimethyl-1H-pyrrole-3-carboxylic acid(2-pyrrolidin-1-yl-ethyl)-amide (Compound 2)

5-Fluoro-1,3-dihydro-indolin-2-one was condensed with 5-formyl-2,4-dimethyl-1H-pyrrole-3-carboxylic acid(2-pyrrolidin-1-yl-ethyl)-amide to yield the title compound. MS+ve APCI 397 [M+1].

### Example 15

### 5-(5-Fluoro-2-oxo-1,2-dihydro-indol-(3Z)-ylidenemethyl)-2,4-dimethyl-1H-pyrrole-3-carboxylic acid(2-ethylamino-ethyl)-amide (Compound 8)

5-Formyl-2,4-dimethyl-1H-pyrrole-3-carboxylic acid(2-ethylamino-ethyl)-amide (99 g), ethanol (400 mL), 5-fluoro-2-oxindole (32 g) and pyrrolidine (1.5 g) were refluxed for 3 hours with stirring. The mixture was cooled to room temperature and the solids collected by vacuum filtration. The solids were stirred in ethanol at 60° C., cooled to room temperature and collected by vacuum filtration. The product was dried under vacuum to give 5-(5-Fluoro-2-oxo-1,2-dihydro-indol-(3Z)-ylidenemethyl)-2,4-dimethyl-1H-pyrrole-3-carboxylic acid(2-ethylamino-ethyl)-amide (75 g, 95% yield). ¹H-NMR (dimethylsulfoxide-d₆) δ 1.03 (t, 3H, CH₃), 2.42, 2.44 (2×s, 6H, 2×CH₃), 2.56 (q, 2H, CH₂), 2.70, 3.30 (2×t, 4H, 2×CH₂), 6.85, 6.92, 7.58, 7.72, 7.76 (5×m, 5H, aromatic, vinyl, and CONH), 10.90 (br s, 1H, CONH), 13.65 (br s, 1H, pyrrole NH). MS m/z 369 [M−1].

### Example 16

### 5-(5-Fluoro-2-oxo-1,2-dihydro-indol-3-ylidenemethyl)-2,4-dimethyl-1H-pyrrole-3-carboxylic acid(2-morpholin-4-yl-ethyl)-amide (Compound 3)

5-Fluoro-1,3-dihydro-indolin-2-one was condensed with 5-formyl-2,4-dimethyl-1H-pyrrole-3-carboxylic acid(2-morpholin-1-yl-ethyl)-amide to yield the title compound.

### BIOLOGICAL EXAMPLES

Methods for MX1 Human Breast Cancer Model

Mice/Husbandry: Female nu/nu mice (Harlan), 13 weeks of age (at pair-match; Day 1), were fed ad libitum water and an irradiated standard rodent diet. Mice were housed in static microisolators on a 12-hour light cycle. The animal care and use program specifically complies with recommendations of the Guide for Care and Use of Laboratory Animals with respect to restraint, husbandry, surgical procedures, feed and fluid regulation, and veterinary care is AAALAC accredited.

Tumor Implantation: Mice were implanted subcutaneously with 1 mm³ MX-1 human breast carcinoma fragments in the flank. Tumors were monitored initially twice weekly, and then daily as the neoplasms reached the desired size, approximately 100 mg. When the carcinomas attained a size between 62-180 mg in calculated tumor weight, the animals were pair-matched into various treatment groups (group mean tumor weights ranged from 99-101 mg). Estimated tumor weight was calculated using the formula:

Tumor Weight(mg)=w²×L divided by 2

where w=width and L=length in mm of a MX-1 carcinoma.

Methods for the MDA-MB-435 Human Breast Cancer Model

1×10⁵ MDA-MB-435 tumor cells were injected into the left cardiac ventricle of female nu/nu mice (n=10). Mice were monitored for weight loss (>20%) and hind limb paralysis as an indicator of bone marrow colonization.

Methods for NCI-H526-Human Small Cell Lung Carcinoma Model

1×10⁵ to 1×10⁶ tumor cells were injected into the subcutaneous region of the hind flank of female nu/nu mice (n=10). Tumor growth was monitored twice a week for 2-4 weeks by caliper measurements.

Methods for LS174t Human Colon Cancer Model

1×10⁵ to 1×10⁶ tumor cells were injected into the subcutaneous region of the hind flank of female nu/nu mice (n=10). Tumor growth was monitored twice a week for 2-4 weeks by caliper measurement.

Methods for HT-29 Human Colon Cancer Model

1×10⁵ to 1×10⁶ tumor cells were injected into the subcutaneous region of the hind flank of female nu/nu mice (n=10). Tumor growth was monitored twice a week for 2-4 weeks by caliper measurement.

### Example 1

### Determination of Enhanced Anti-Tumor Efficacy of Compound 1 in Combination with Docetaxel in the MX-1 Human Breast Carcinoma Subcutaneous Tumor Model

This examples shows the evaluation of the effects of combined treatment of compound 1 and Docetaxel on efficacy and toxicity in a human breast cancer model.

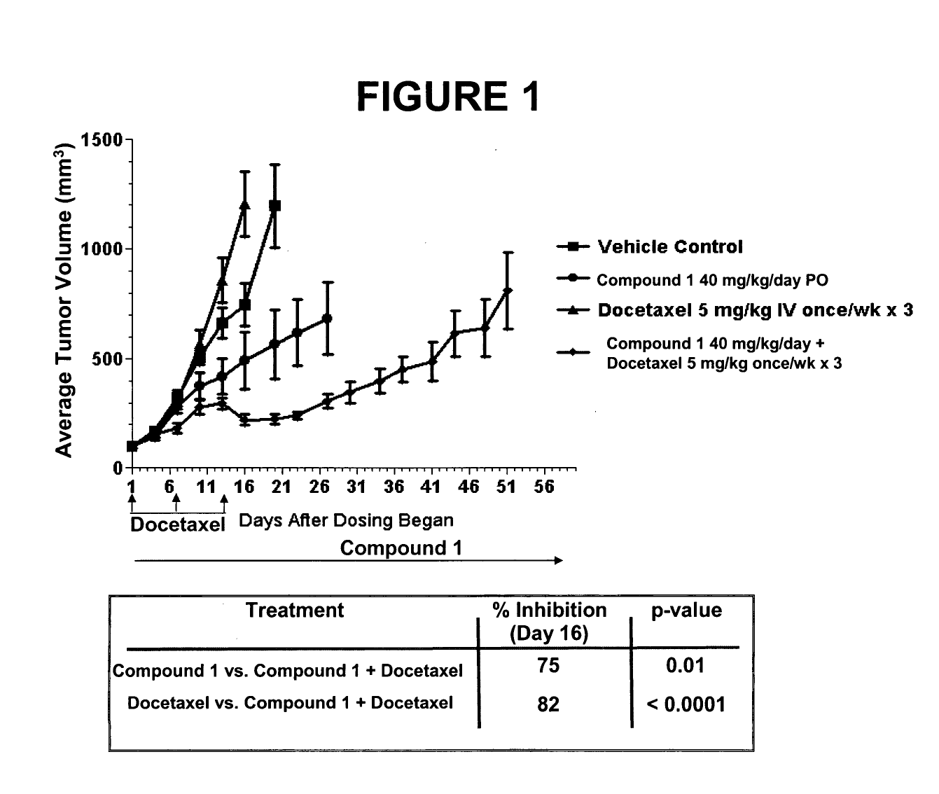

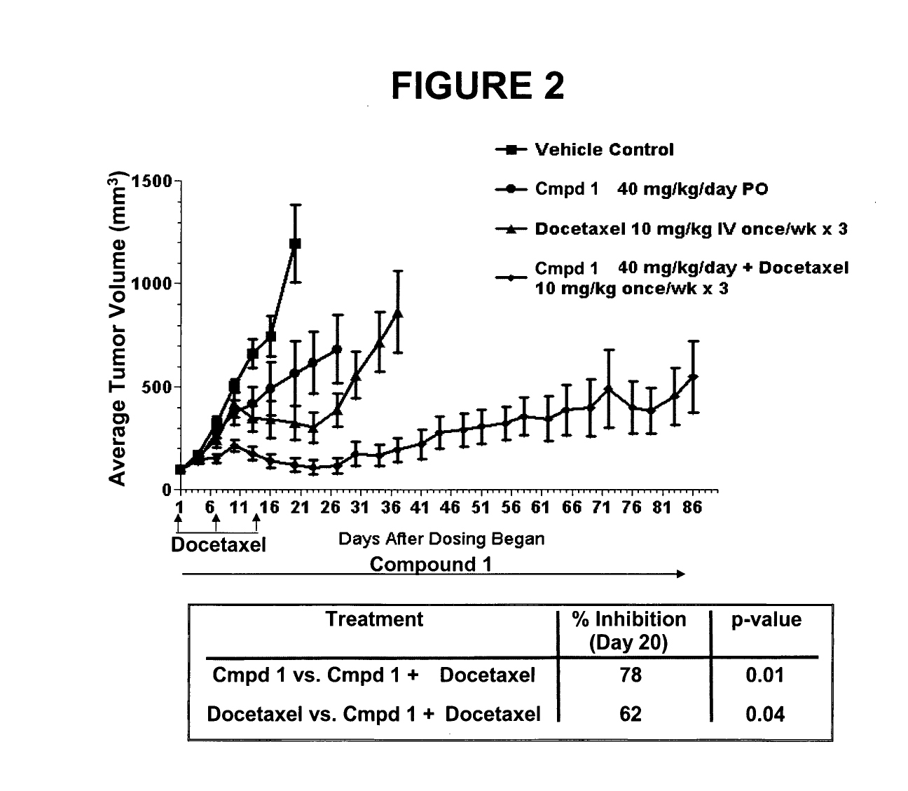

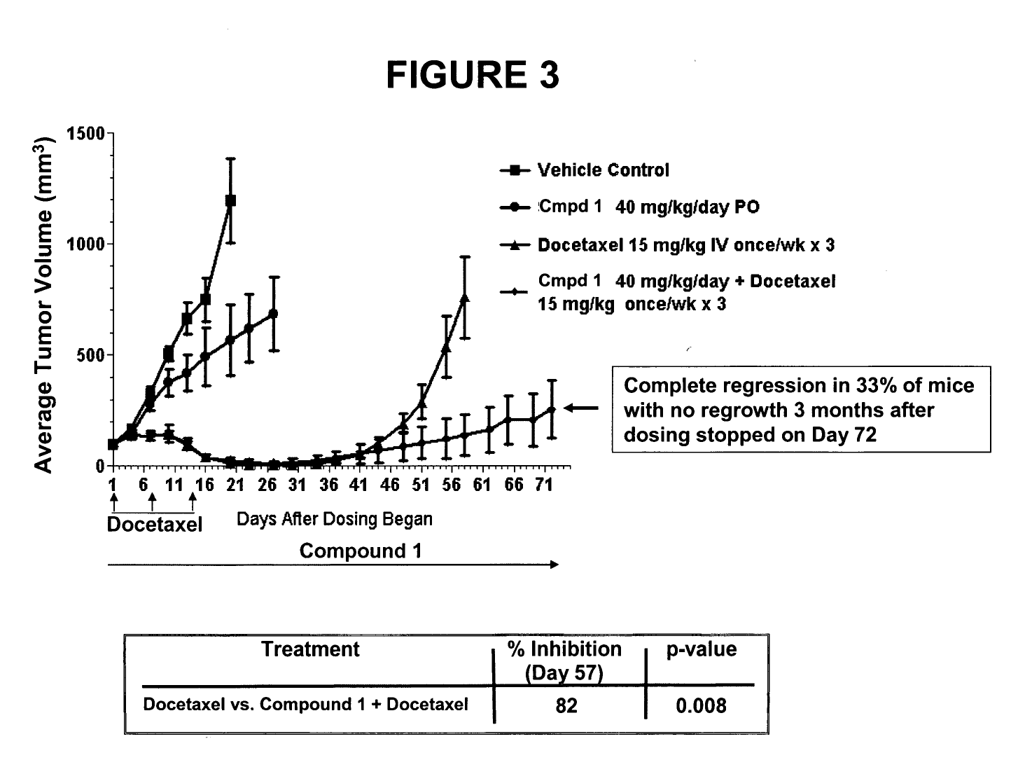

Tumors were grown to a volume of approximately 100 mm³ prior to dosing. Table 1 is a compilation of the data obtained using this model (see also FIGS. 1-3).

TABLE 1

| | | Dose | | | | | | |
|---|---|---|---|---|---|---|---|---|
| | | (mg/kg)/ | | % | P | | % | P |
| Rte | Compound | schedule | Day | Inhibition | value* | Day | Inhibition | value* |
| PO | CMC | QD to | — | — | — | — | — | — |
| | | end | | | | | | |
| IV | Saline | QWK × 3 | — | — | — | — | — | — |
| PO | Compound 1 | 40 QD | 20 | 53 | 0.02 | — | — | — |
| | | to end | | | | | | |
| IV | Docetaxel | 5 QWK × 3 | 16 | 0 | NS | — | — | — |
| IV | Docetaxel | 10 | 16 | 60 | 0.005 | — | — | — |
| | | QWK × 3 | | | | | | |
| IV | Docetaxel | 15 | 16 | 95 | <0.0001 | — | — | — |
| | | QWK × 3 | | | | | | |
| PO/IV | Compound 1/ | 40 QD | 16 | Vs | 0.01 | 27 | Vs | 0.04 |
| | Docetaxel | to end/5 | | Compound 1: | <0.0001 | | Compound 1: | |
| | | QWK × 3 | | 75 | | | 55 | |
| | | | | Vs | | | | |
| | | | | Docetaxel: | | | | |
| | | | | 82 | | | | |
| PO/IV | Compound 1/ | 40 QD | 20 | Vs | 0.01 | 37 | Vs | 0.005 |
| | Docetaxel | to end/ | | Compound 1: | 0.04 | | Docetaxel: | |
| | | 10 | | 78 | | | 77 | |
| | | QWK × 3 | | Vs | | | | |
| | | | | Docetaxel: | | | | |
| | | | | 62 | | | | |
| PO/IV | Compound 1/ | 40 QD | 57 | Vs | 0.008 | — | — | — |
| | Docetaxel | to end/ | | Docetaxel: | | | | |
| | | 15 | | 82 | | | | |
| | | QWK × 3 | | | | | | |

CMC = Carboxymethyl cellulose
QD = every day
QWK = once every week
N/A = Not Applicable;
NS = Not Significant
*Comparisons for Student's t-test

In the MX-1 human breast carcinoma subcutaneous tumor model, Compound 1 treatment resulted in 52% inhibition (p=0.02) on Day 20 when delivered orally as a monotherapy at 40 mg/kg/day. Docetaxel treatment resulted in a dose response effect, with no efficacy at 5 mg/kg, 60% inhibition at 10 mg/kg (p=0.005) and 95% inhibition at 15 mg/kg (p<0.0001) 16 days after dosing. After Docetaxel administration was complete in the 10 and 15 mg/kg treated groups, the tumors regrew with slopes similar to those of the vehicle control groups.

The combination of daily dosing of Compound 1 with 5 mg/kg Docetaxel once a week for three weeks resulted in markedly enhanced inhibition of tumor growth relative to the non-effective 5 mg/kg Docetaxel and relative to 40 mg/kg/day of Compound 1 (Day 27: 55% inhibition, p=0.04).

The combination of daily dosing of Compound 1 with 10 mg/kg Docetaxel once a week for three weeks resulted in markedly enhanced inhibition of tumor growth over Docetaxel alone (Day 20: 62% inhibition, p=0.04; Day 37: 77% inhibition, p=0.005) or Compound 1 alone (Day 20: 78% inhibition, p=0.01).

Maintenance model: The combination of Compound 1 with 15 mg/kg Docetaxel once a week for three weeks resulted in markedly enhanced delay of tumor growth after Docetaxel administration ceased as compared to the regrowing tumors in mice treated with Docetaxel alone (Day 57: 82% inhibition, p=0.008).

The combination of Compound 1 and Docetaxel was well-tolerated in these studies.

### Example 2

### Study of MX-1 Breast Cancer Efficacy Study (Compound 1 & Docetaxel)-Determination of Enhanced Anti-Tumor Efficacy of Compound 1 in Combination with Docetaxel in the MX-1 Human Breast Carcinoma Subcutaneous Tumor Model

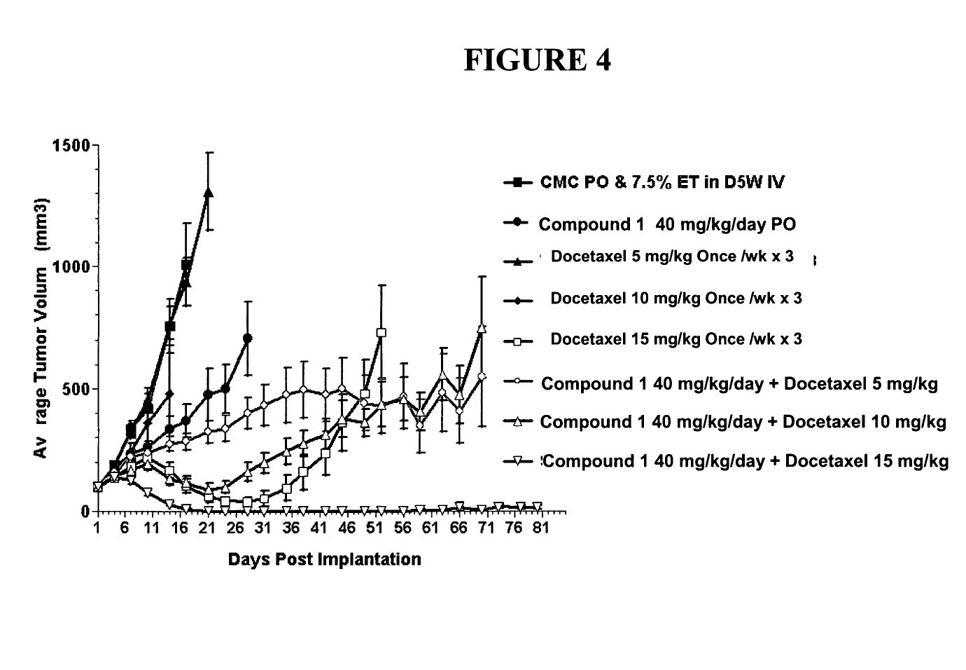

This example evaluates the effects of combined treatment of Compound 1 and Docetaxel on efficacy and toxicity in a human breast cancer model. Tumors were grown to a volume of approximately 100 mm³ prior to dosing. Table 2 is a compilation of the data obtained using this model (see also FIG. 4).

TABLE 2

| | | Dose (mg/kg)/ | | | |
|---|---|---|---|---|---|
| Rte | Compound | schedule | Day | % Inhibition | P value* |
| PO/ | CMC | QD to end | N/A | N/A | N/A |
| IV | Saline | qwk × 3 | | | |
| PO | Compound 1 | 40 QD to end | 17 | 63 | <0.0001 |
| IV | Docetaxel | 5 QWK × 3 | 17 | NS | NS |
| IV | Docetaxel | 10 QWK × 3 | 14 | 36 | NS |
| IV | Docetaxel | 15 QWK × 3 | 17 | 90 | <0.0001 |
| PO/ | Compound | 40 QD to end/ | 28 | vs | 0.08 (NS) |
| IV | 1/Docetaxel | 5 QWK × 3 | 21 | Compound 1: | <0.0001 |
| | | | | 43 | |
| | | | | vs Docetaxel: | |
| | | | | 75 | |
| PO/ | Compound | 40 QD to end/ | 28 | vs | 0.002 |
| IV | 1/Docetaxel | 10 QWK × 3 | 14 | Compound 1: | 0.10 (NS) |
| | | | | 77 | |
| | | | | vs Docetaxel: | |
| | | | | 72 | |
| PO/ | Compound | 40 QD to end/ | 52 | vs Docetaxel & | <0.0001 |
| IV | 1/Docetaxel | 15 QWK × 3 | | Compound 1: | |
| | | | | 100 | |

QD = every day
QWK = once every week
N/A = Not Applicable;
NS = Not Significant
*Comparisons for Student's t-test

In the MX-1 human breast carcinoma sc tumor model, Compound 1 treatment resulted in 63% inhibition (p<0.0001) on Day 17 when delivered orally as a monotherapy at 40 mg/kg/day. Docetaxel treatment resulted in a dose response effect, with no efficacy at 5 mg/kg, 36% inhibition at 10 mg/kg (Day 14) and 90% inhibition at 15 mg/kg (p <0.0001) 17 days after dosing. After Docetaxel administration was complete in the 15 mg/kg treated groups, the tumors regrew with a slope similar to those of the vehicle control group.

The combination of daily dosing of Compound 1 with 5 mg/kg Docetaxel once a week for three weeks resulted in markedly enhanced inhibition of tumor growth relative to the non-effective 5 mg/kg Docetaxel and relative to 40 mg/kg/day of Compound 1 (Day 28: 43% inhibition, p=0.08-trending toward significance).

The combination of daily dosing of Compound 1 with 10 mg/kg Docetaxel once a week for three weeks resulted in markedly enhanced inhibition of tumor growth over Docetaxel or Compound 1 alone (Day 28: 77% inhibition, p=0.002).

Maintenance model: The combination of Compound 1 with 15 mg/kg Docetaxel once a week for three weeks resulted in marked tumor regression as compared to the regrowing tumors in mice treated with Docetaxel alone (Day 52: 100% inhibition, p<0.0001). The combination of Compound 1 and Docetaxel was well-tolerated in these studies.

### Example 3

### MX-1 Breast Cancer Efficacy Study (Compound 1 & 5-Flurouracil)-Determination of Enhanced Anti-Tumor Efficacy of Compound 1 in Combination with 5-Flurouracil (5-FU) in the MX-1 Human Breast Carcinoma Subcutaneous Tumor Model

This example evaluates the effects of combined treatment of Compound 1 and 5-FU on efficacy and toxicity in a human breast cancer model

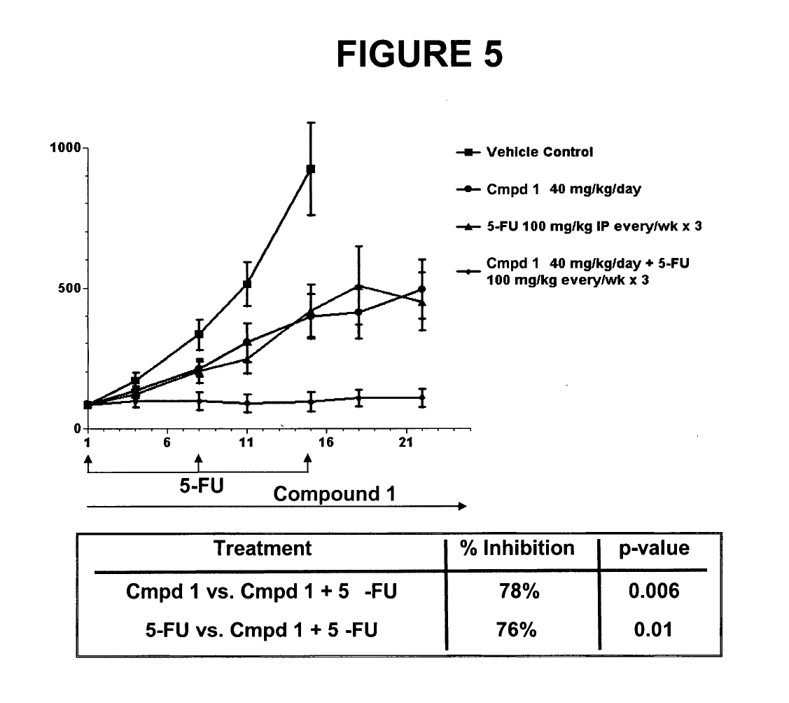

Tumors were grown to a volume of approximately 100 mm³ prior to dosing. Table 3 shows the results obtained with this model (see FIG. 5).

TABLE 3

| | | Dose (mg/kg)/ | | | P |
|---|---|---|---|---|---|
| Rte | Compound | schedule | Day | % Inhibition | value* |
| PO/IV | CMC | QD to end | N/A | N/A | N/A |
| | | QWK × 3 | | | |
| PO | Compound 1 | 40 QD to end | 15 | 57 | 0.01 |
| IV | 5-FU | 100 QWK × 3 | 15 | 45 | 0.02 |
| PO/IV | Compound | 40 QD to | 22 | vs Compound | 0.006 |
| | 1/5-FU | end/QWK × 3 | | 1: 78 | 0.01 |
| | | | | vs 5-FU: 76 | |

QD = every day
QWK = once every week
N/A = Not Applicable;
NS = Not Significant
*Comparisons for Student's t-test

The combination therapy of oral administration of Compound 1 at 40 mg/kg/day with the chemotherapy drug 5-Fluorouracil (5-FU) administered i.p. at 100 mg/kg once a week for three weeks resulted in a significant tumor growth inhibition compared to each agent administered as a monotherapy: 78% inhibition (p=0.006) on day 22 as compared to Compound 1 alone and 76% inhibition (p=0.01) on day 22 as compared to 5-FU alone. Clinically, 5-FU is administered orally as the prodrug Capecitabine.

### Example 4

### MX-1 Breast Cancer Efficacy Study (Compound 1 & Doxorubicin Hydrochloride)-Determination of Enhanced Anti-Tumor Efficacy of Compound 1 in Combination with Doxorubicin Hydrochloride in the MX-1 Human Breast Carcinoma Subcutaneous Tumor Model

This example evaluates the effects of combined treatment of Compound 1 and Doxorubicin Hydrochloride on efficacy and toxicity in a human breast cancer model.

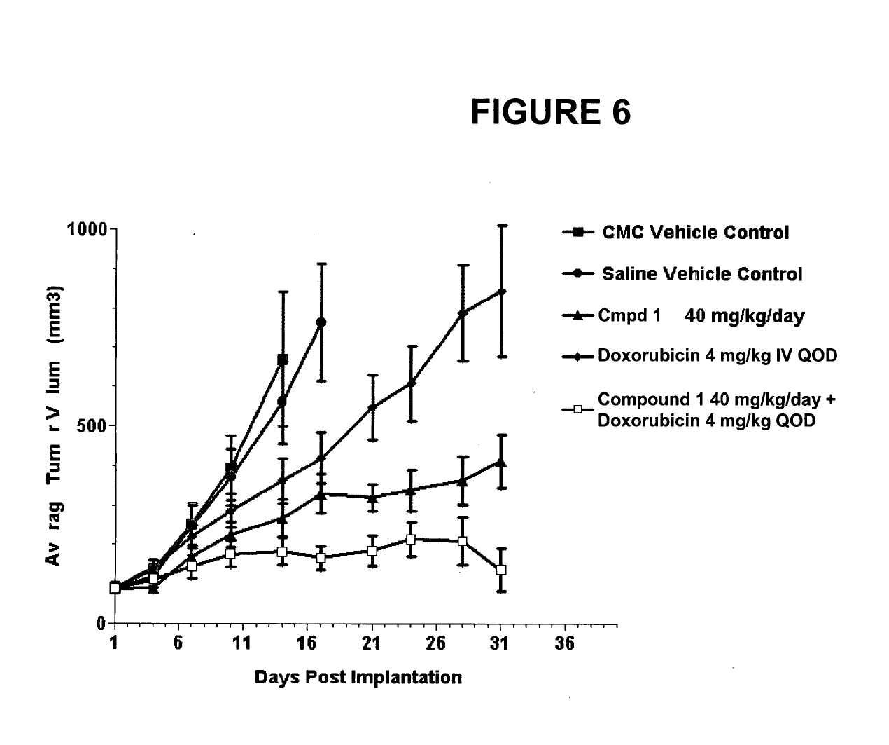

Tumors were grown to a volume of approximately 100 mm³ prior to dosing. Table 4 shows the results obtained with this model. (see FIG. 6).

TABLE 4

| | | Dose | | | |
|---|---|---|---|---|---|
| | | (mg/kg)/ | | | P |
| Rte | Compound | schedule | Day | % Inhibition | value* |
| PO | CMC | QD to | N/A | N/A | N/A |
| | | end | | | |
| PO | Compound 1 | 40 QD to | 14 | 62 | 0.03 |
| | | end | | | |
| IV | Doxorubicin | 4 QOD × 3 | 14 | 48 | 0.07 |
| | Hydrochloride | | | | (NS) |
| PO/ | Compound 1/ | 40 QD to | 31 | vs Compound 1: | 0.01 |
| IV | Doxorubicin | end/ | | 60 | 0.001 |
| | Hydrochloride | 4 QOD × 3 | | vs Docetaxel: 81 | |

QD = every day
QOD = every other day
N/A = Not Applicable;
NS = Not Significant
*Comparisons for Student's t-test

The combination therapy of oral administration of Compound 1 at 40 mg/kg/day with the chemotherapy drug Doxorubicin Hydrochloride administered i.p. at 4 mg/kg once every other day for three doses resulted in a significant MX1 tumor growth inhibition compared to each agent administered as a monotherapy: 60% inhibition (p=0.01) on day 31 as compared to Compound 1 alone and 81% inhibition (p=0.001) on day 31 as compared to 5-FU alone.

### Example 5

### NCI-H526 Small Cell Lung Cancer Efficacy Study (Compound 1 & Cisplatin)

NCI-H526 SCLC cells were cultured using standard technique in RPMI 1640 supplemented with 10% fetal bovine serum, 2 mM glutamine, 1 mM sodium pyruvate (Life Technologies Inc., Gaithersburg, Md.), and maintained routinely in a humidified chamber at 37° C. and 5% carbon dioxide.

Cells to be implanted in mice were harvested from cell culture flasks during exponential growth, washed once with sterile phosphate-buffered saline (PBS), counted, and resuspended in PBS to a suitable concentration prior to implantation.

All animal studies were carried out in an AAALAC, International accredited animal facility and in accordance with the Institute of Laboratory Animal Research (National Institutes of Health, Bethesda, Md.) Guide for the Care and Use of Laboratory Animals. Nine to twelve week old female athymic nu/nu mice purchased from Charles River Laboratories (Wilmington, Mass.) were used.

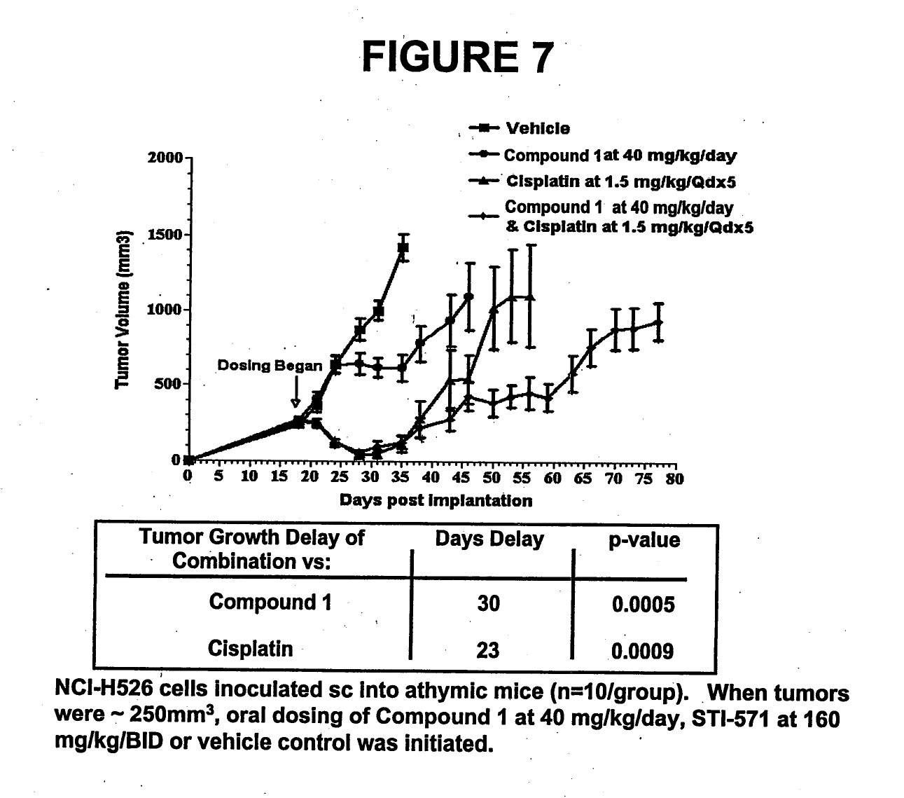

Mice received subcutaneous injections into the hind flank on Day 0 with 5×10⁶ NCI-H526 cells. Subcutaneous tumor-bearing athymic mice (250-300 mm³ tumor volume) were treated either PO once daily with Compound 1 to the end of the study, intraperitoneally once daily with Cisplatin for the first 5 days, or a combination of the two. Cisplatin was prepared in 0.9% saline. Compounds or their vehicles were administered as indicated in TABLE 5. Tumors were established between 250-300 mm³ when dosing began on day 18 after cell implantation. Tumor growth was measured twice weekly using Vernier calipers for the duration of the treatment. Tumor volumes were calculated as the product of length× width× height. For all studies, p-values were calculated using the two-tailed Student's t test. Table 5 shows the results obtained with this model (see FIG. 7).

TABLE 5

| | | | | Day tumor | |
|---|---|---|---|---|---|
| E#/ | | Dose | | reached | |
| Route | Compound | (mg/kg) | Regimen | 900 mm³ | P value* |
| 2958/PO | Compound 1 | 40 | QD | 41 | 0.0005 |
| IP | Cisplatin | 1.5 | QD× 5 | 48 | 0.0009 |
| PO/IP | Compound 1 | 40 | QD | 70 | <0.0001 |
| | Cisplatin | 1.5 | QD× 5 | | 0.004 |
| PO | Vehicle | N/A | QD | 30 | N/A |

*Student's t-test, two-tailed
N/A = Not Applicable
QD = every day

In the KIT-positive NCI-H526 SCLC tumor xenograft model, daily oral administration of Compound 1 at 40 mg/kg in combination with Cisplatin administered i.p. at 1.5 mg/kg for the first five days resulted in a 29 day delay of tumor growth to reach 900 mm³, compared to Compound 1 monotherapy and 22 days compared to cisplatin monotherapy (p<0.0001 and p=0.004, respectively).

### Example 6

### MDA-MB-435 Breast Cancer Efficacy Study (Compound 1 & Docetaxel)-Determination of Enhanced Anti-Tumor Efficacy of Compound 1 in Combination with Docetaxel in the MDA-MB-435 Orthotopic Human Breast Carcinoma Bone Marrow Colonization Model

This example evaluates the effects of combined treatment of Compound 1 and Docetaxel on efficacy and toxicity in a human breast cancer model. Efficacy is indicated by improved survival, which, in turn, is indicated by hind-limb paralysis or weight loss (>20%) due to bone marrow colonization of tumor cells.

Tumor cells were injected into the mammary fat pad of female nu/nu mice and mice were monitored for weight loss (>20%) and hind limb paralysis as an indicator of bone marrow colonization of tumor cells.

Tumor cells were injected into the left cardiac ventricle of female nu/nu mice and mice were monitored for weight loss (>20%) and hind limb paralysis as an indicator of bone marrow colonization

TABLE 6

| | | Dose | | Day of | |
|---|---|---|---|---|---|
| | | (mg/kg)/ | Median | Statistical | |
| Rte | Compound | Schedule | Survival | Analysis | P value* |
| PO | CMC | QD to end | 46 | 55 | N/A |
| PO | Compound 1 | 40 QD to | 52 | 55 | 0.03 |
| | | end | | | |
| IP | Docetaxel | 5 QWK × 3 | 52 | 55 | 0.3 (NS) |
| PO/IV | Compound | 40 QD to | 60 | 55 | 0.017 |
| | 1/Docetaxel | end/5 | | | (v cmpd 1) |
| | | QWK × 3 | | | 0.0006 (vs |
| | | | | | Docetaxel) |

QD = every day
QWK = once every week
N/A = Not Applicable;
NS = Not Significant
*Comparisons for Student's t-test

In the MDA-MB-435 orthotopic human breast carcinoma tumor model, Compound 1 treatment resulted in significant improvement in mouse survival when delivered as a monotherapy at 40 mg/kg/day compared to vehicle treatment alone (52 vs. 46 days, p=0.03). Treatment with Docetaxel as a monotherapy at 5 mg/kg/week for 3 weeks did not significantly improve survival compared to vehicle treated mice (52 vs. 46 days, p=0.3).

The combination of Compound 1 at 40 mg/kg/day and Docetaxel at 5 mg/kg/week for 3 weeks resulted in significantly enhanced survival compared with Compound 1 (median survival 60 vs. 52 days, p=0.017) or Docetaxel (median survival 60 vs. 52 days, p=0.0006) as monotherapies.

### Example 7

### LS174t Colon Cancer Efficacy Study (Compound 1 & CPT-11)-Determination of Enhanced Anti-Tumor Efficacy of Compound 1 in Combination with CPT-11 (Irinotecan) in the LS174t Human Colon Carcinoma Subcutaneous Tumor Model

This example evaluates the effects of combined treatment of Compound 1 and CPT-11 (Irinotecan) on efficacy and toxicity in a human colon cancer model

Tumors were grown to a volume of approximately 100 mm³ prior to dosing. Table 7 shows the results obtained with this model.

TABLE 7

| | | Dose | | | |
|---|---|---|---|---|---|
| | | (mg/kg)/ | | % | P |
| Rte | Compound | schedule | Day | Inhibition | value* |
| PO | CMC | QD to end | 23 | N/A | N/A |
| IP | D5W | QWK × 3 | 23 | N/A | N/A |
| PO/IP | CMC/D5W | QD to end/ | 23 | N/A | N/A |
| | | QWK × 3 | | | |
| PO | Compound 1 | 20 QD to end | 23 | 34.9 | 0.19 |
| | | | | | (NS) |
| PO | Compound 1 | 40 QD to end | 23 | 67.9 | 0.004 |
| IP | CPT-11 | 100 QWK × 3 | 23 | 63.6 | 0.008 |
| PO/IV | Compound 1/ | 20 QD to | 23 | vs veh: 82.7 | 0.0003 |
| | CPT-11 | end/QWK × 3 | | vs Cmpd 1: | 0.014 |
| | | | | 73.4 | 0.06 |
| | | | | vs CPT-11: | (NS) |
| | | | | 50.9 | |
| PO/IV | Compound 1/ | 40 QD to | 23 | vs veh: 88.7 | 0.00002 |
| | CPT-11 | end/QWK × 3 | | vs Cmpd 1: | 0.07 |
| | | | | 64.6 | (NS) |
| | | | | vs CPT-11: | 0.02 |
| | | | | 67.9 | |

QD = every day
QWK = once every week
D5W = 5% dextrose in water
N/A = Not Applicable;
NS = Not Significant
*Comparisons for Student's t-test

In the LS174t human colon carcinoma subcutaneous tumor model, Compound 1 treatment resulted in significant (68% at day 23) inhibition of tumor growth when delivered as a monotherapy at 40 mg/kg/day. Treatment with compound 1 at 20 mg/kg/day as a monotherapy did not result in significant growth inhibition in this model. Treatment with CPT-11 (Ironotecan) as a monotherapy at 100 mg/kg/week for 3 weeks also resulted in significant tumor growth inhibition (64% at day 23). Over the duration of the study, Compound 1 markedly inhibited growth of tumors as a monotherapy at 40 mg/kg/day while tumors treated with 20 mg/kg/day or CPT-11 at 100 mg/kg/week for 3 weeks grew at a slightly slower rate compared to vehicle treated controls.

The combination of Compound 1 at 20 or 40 mg/kg/day and CPT-11 at 100 mg/kg/week for 3 weeks resulted in enhanced inhibition of tumor growth relative to Compound 1 or CPT-11 as a monotherapy within the first 3 weeks of the study. The combination of Compound 1 at the sub-optimal dose of 20 mg/kg/day and CPT-11 at 100 mg/kg/week for 3 weeks resulted in enhanced inhibition of tumor growth relative to Compound 1 or CPT-11 alone (Day 23: Compound 1: 74% inhibition, p=0.014 and CPT-11 51% inhibition; p=0.06-trend toward significance). In addition, the combination of Compound 1 at 40 mg/kg/day and CPT-11 at 100 mg/kg/week for 3 weeks resulted in enhanced inhibition of tumor growth relative to Compound 1 or CPT-11 alone (Day 23: Compound 1: 65% inhibition, p=0.07; CPT-11: 68% inhibition, p=0.02). The combination of Compound 1 with CPT-11 was well tolerated in these studies.

### Example 8

### HT-29 Colon Cancer Efficacy Study (Compound 1 & CPT-11)-Determination of Enhanced Anti-Tumor Efficacy of Compound 1 in Combination with CPT-11 (Irinotecan) in the HT-29 Human Colon Carcinoma Subcutaneous Tumor Model

This example evaluates the effects of combined treatment of Compound 1 and CPT-11 (Irinotecan) on efficacy and toxicity in an additional human colon cancer model.

Tumors were grown to a volume of approximately 100 mm³ prior to dosing. Table 8 shows the results obtained with this model.

TABLE 8

| | | Dose | | | |
|---|---|---|---|---|---|
| | | (mg/kg)/ | | % | P |
| Rte | Compound | Schedule | Day | Inhibition | value* |
| PO | CMC | QD to end | 38 | N/A | N/A |
| IP | D5W | QWK × 3 | 38 | N/A | N/A |
| PO/IP | CMC/D5W | QD to end/ | 38 | N/A | N/A |
| | | QWK × 3 | | | |
| PO | Compound 1 | 20 QD to end | 38 | 77.8 | 0.002 |
| IP | CPT-11 | 100 QWK × 3 | 38 | 43.6 | 0.18 |
| | | | | | (NS) |
| PO/IV | Compound 1/ | 20 QD to | 38 | vs veh: 87.4 | 0.001 |
| | CPT-11 | end/QWK × 3 | | vs Cmpd 1: | 0.04 |
| | | | | 42.4 | 0.02 |
| | | | | vs CPT-11: | |
| | | | | 71.4 | |

QD = every day
QWK = once every week
D5W = 5% dextrose in water
N/A = Not Applicable;
NS = Not Significant
*Comparisons for Student's t-test

In the HT-29 human colon carcinoma subcutaneous tumor model, Compound 1 treatment resulted in significant (78% at day 38) inhibition of tumor growth when delivered as a monotherapy at 20 mg/kg/day. Treatment with CPT-11 (Irinotecan) as a monotherapy at 100 mg/kg/week for 3 weeks displayed a trend toward tumor growth inhibition, but growth inhibition was not significant. Over the duration of the study, Compound 1 and CPT-11 treatment each exhibited an overall trend towards slowing growth of tumors as monotherapies.

The combination of Compound 1 at 20 and CPT-11 at 100 mg/kg/week for 3 weeks resulted in significantly enhanced inhibition of tumor growth relative to Compound 1 (Day 38: 42% inhibition, p=0.04) or CPT-11 (Day 38: 71% inhibition, p=0.02) as monotherapies. In addition, the combination of compound 1 and CPT-11 exhibited a trend toward marked growth inhibition and survival advantage over the duration of the studies compared to either monotherapy. The combination of Compound 1 with CPT-11 was well tolerated in these studies.

It will be apparent to those skilled in the art that various modifications and variations can be made in the methods and compositions of the present invention without departing from the spirit or scope of the invention. Thus, it is intended that the present invention covers the modifications and variations of this invention provided they come within the scope of the appended claims and their equivalents.

What is claimed is:

1. A method of treating cancer comprising administering to a patient in need thereof an effective amount of a compound of Formula I: 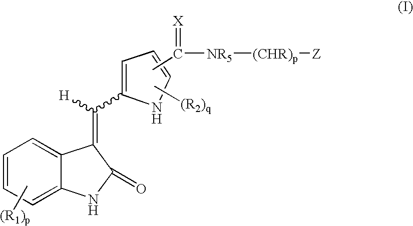
wherein,
each R is independently hydrogen, hydroxy, alkyl, aryl, cycloalkyl, heteroaryl, alkoxy, heterocyclic or amino;
each R₁ is independently alkyl, halo, alkoxy, haloalkyl, haloalkoxy, cycloalkyl, heterocyclic, hydroxy, —C(O)—R₈, —NR₉R₁₀, —NR₉C(O)—R₁₂ or —C(O)NR₉R₁₀;
each R₂ is independently alkyl, aryl, heteroaryl, —C(O)—R₈ or SO₂R″, where R″ is alkyl, aryl, heteroaryl, NR₉N₁₀ or alkoxy;
each R₅ is independently hydrogen, alkyl, aryl, haloalkyl, cycloalkyl, heteroaryl, heterocyclic, hydroxy, —C(O)—R₈ or (CHR)ᵣR₁₁;
X is O or S;
j is 0 or 1; l
p is 0, 1, 2 or 3;
q is 0, 1 or 2;
r is 0, 1, 2 or 3;
R₈ is hydroxy, alkyl, aryl, heteroaryl, alkoxy, cycloalkyl or heterocyclic;
R₉ and R₁₀ are independently hydrogen, alkyl, aryl, aminoalkyl, heteroaryl, cycloalkyl and heterocyclic, or R₉ and R₁₀ together with N may form a ring, where the ring atoms are selected from the group consisting of C, N, O and S;
R₁₁, is hydroxy, amino, monosubstituted amino, disubstituted amino, alkyl, aryl, heteroaryl, alkoxy, cycloalkyl or heterocyclic
R₁₂ is alkyl, aryl, heteroaryl, alkoxy, cycloalkyl or heterocyclic; and
Z is hydroxy, —O-alkyl, or —NR₃R₄, where R₃ and R₄ are independently hydrogen, alkyl, aryl, heteroaryl, cycloalkyl, or heterocyclic, or R₃ and R₄ may combine with N to form a ring where the ring atoms are selected from the group consisting of CH₂, N, O and S, or 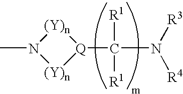
wherein Y is independently CH₂, O, N or S, Q is C or N, n is independently 0, 1, 2, 3 or 4, and m is 0, 1, 2 or 3;
or a pharmaceutically acceptable salt, hydrate or solvate thereof, in combination with at least one chemotherapeutic agent selected from the group consisting of microtubule interference agents, topoisomerase inhibitors, alkylating agents, thymidylate synthase inhibitors, irreversible steroidal aromatase inactivators, anti-metabolites, pyrimidine antagonists, purine antagonists, ribonucleotide reductase inhibitors, and kinase inhibitors.

2. The method of claim 1, wherein R₁ is halo and p is 1.

3. The method of claim 1, wherein R₁ is F or Cl and p is 1.

4. The method of claim 1, wherein Z is —NR₃R₄ wherein R₃ and R₄ are lower alkyl or form a morpholine ring.

5. The method of claim 1, wherein Z is: 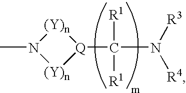
wherein each Y is CH₂, each n is 2, m is 0 and R₃ and R₄ form a morpholine ring.

6. The method of claim 1, wherein R₂ is methyl and q is 2, wherein the methyls are bonded at the 3 and 5 positions.

7. The method of claim 1, wherein the compound of formula I is selected from the group consisting of 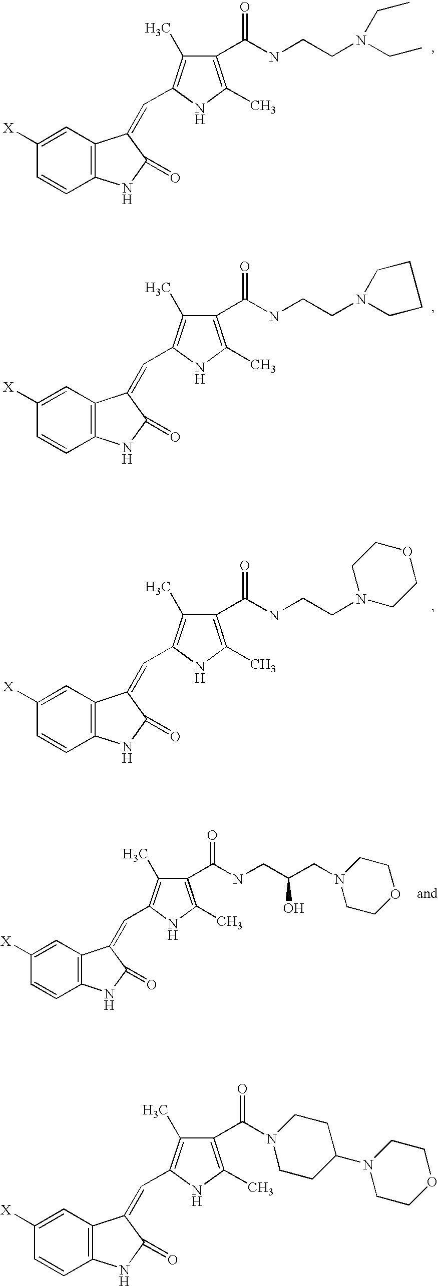
and pharmaceutically acceptable salts, solvates and hydrates thereof.

8. The method of claim 1, wherein the compound of formula I is selected from the group consisting of: 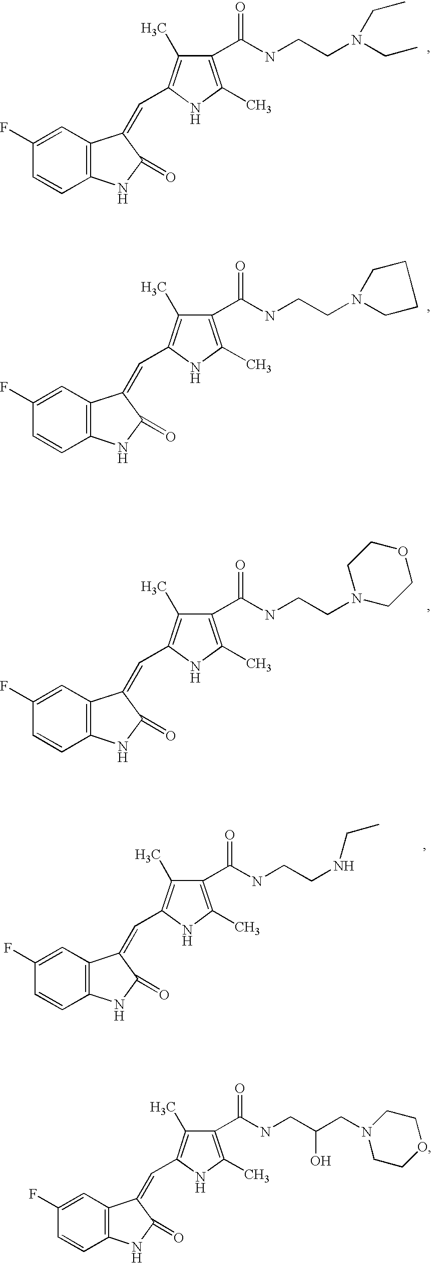 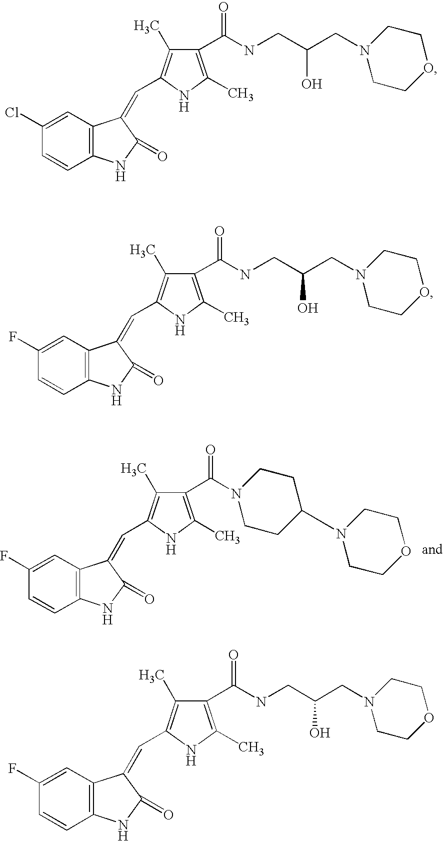
and pharmaceutically acceptable salts, solvates and hydrates thereof.

9. The method of claim 1, wherein the compound of Formula (I) is: 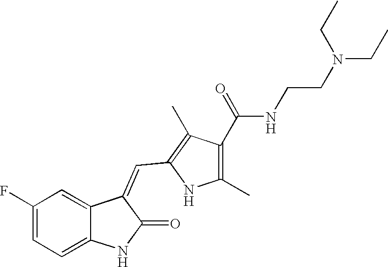
or a pharmaceutically acceptable salt, solvate or hydrate thereof.

10. The method of claim 9, wherein the salt is a malate salt.

11. The method of claim 1, wherein the at least one chemotherapeutic agent is selected from the group consisting of taxanes, vinca alkyloids, topoisomerase I inhibitors and topoisomerase II inhibitors.

12. The method of claim 1, wherein the at least one chemotherapeutic agent is selected from the group consisting of paclitaxel, docetaxel, vinblastine, vincristine, vindesine, irinotecan, doxorubicin, epirubicin, leucovorin, etopside, teniposide, idarubicine, gemcitabine, daunorubicin, carboplatin, cisplatin, oxaliplatin, chlorambucil, melphalan, cyclophosphamide, ifosfamide, temozolomide, thiotepa, mitomycin C, busulfan, carmustine, lomustine, 5-fluorouracil, capecitabine, exemestane, methotrexate, trimetrexate, fluorouracil, fluorodeoxyuridine, azacytidine, mercaptopurine, thioguanine, pentostatin, cytarabine, fludarabine, hydroxyurea, bevacizumab, cetuximab, gefitinib and imatinib.

13. The method of claim 1, wherein the cancer is breast cancer, small cell lung carcinoma, colon cancer, non-small cell lung cancer, renal cell cancer, a gastrointestinal stromal tumor, thyroid cancer, a sarcoma or a neuroendocrine tumor.

14. The method of claim 1, wherein the cancer is non-small cell lung cancer and the at least one chemotherapeutic agent is carboplatin and paclitaxel.

15. The method of claim 1, wherein the cancer is non-small cell lung cancer and the at least one chemotherapeutic agent is carboplatin, taxotere, cisplatin, gemcitabine, 5-fluorouracil, irinotecan or leucovorin.

16. The method of claim 1, wherein the cancer is colon cancer and the at least one chemotherapeutic agent is 5-fluorouracil, oxaliplatin or leucovorin.

17. A method of treating cancer comprising administering to a patient in need thereof an effective amount of a compound selected from the group consisting of:
5-(5-Fluoro-2-oxo-1,2-dihydro-indol-3-ylidenemethyl)-2,4-dimethyl-1H-pyrrole-3-carboxylic acid(2-diethylamino-ethyl)-amide;
5-(5-Fluoro-2-oxo-1,2-dihydro-indol-3-ylidenemethyl)-2,4-dimethyl-1H-pyrrole-3-carboxylic acid(2-pyrrolidin-1-yl-ethyl)-amide;
5-(5-Fluoro-2-oxo-1,2-dihydro-indol-3-ylidenemethyl)-2,4-dimethyl-1H-pyrrole-3-carboxylic acid(2-morpholin-4-yl-ethyl)-amide;
(S)-5-(5-Fluoro-2-oxo-1,2-dihydro-indol-3-ylidenemethyl)-2,4-dimethyl-1H-pyrrole-3-carboxylic acid(2-hydroxy-3-morpholin-4-yl-propyl)-amide;
(R)-5-(5-Fluoro-2-oxo-1,2-dihydro-indol-3-ylidenemethyl)-2,4-dimethyl-1H-pyrrole-3-carboxylic acid(2-hydroxy-3-morpholin-4-yl-propyl)-amide;
5-(5-Fluoro-2-oxo-1,2-dihydro-indol-3-ylidenemethyl)-2,4-dimethyl-1H-pyrrole-3-carboxylic acid(2-hydroxy-3-morpholin-4-yl-propyl)-amide;
5-(5-Chloro-2-oxo-1,2-dihydro-indol-3-ylidenemethyl)-2,4-dimethyl-1H-pyrrole-3-carboxylic acid(2-hydroxy-3-morpholin-4-yl-propyl)-amide;
5-(5-Fluoro-2-oxo-1,2-dihydro-indol-3-ylidenemethyl)-2,4-dimethyl-1H-pyrrole-3-carboxylic acid(2-ethylamino-ethyl)-amide; and
3-[3,5-dimethyl-4-(4-morpholin-4-yl-piperidine-1-carbonyl)-1H-pyrrol-2-methylene]-5-fluoro-1,3-dihydro-indol-2-one,
or a pharmaceutically acceptable salt, hydrate or solvate thereof, in combination with at least one chemotherapeutic agent selected from the group consisting of microtubule interference agents, topoisomerase inhibitors, alkylating agents, thymidylate synthase inhibitors, irreversible steroidal aromatase inactivators, anti-metabolites, pyrimidine antagonists, purine antagonists, ribonucleotide reductase inhibitors, and kinase inhibitors.

18. The method of claim 17, wherein the at least one chemotherapeutic agent is selected from the group consisting of taxanes, vinca alkyloids, topoisomerase I inhibitors and topoisomerase II inhibitors.

19. The method of claim 17, wherein the at least one chemotherapeutic agent is selected from the group consisting of paclitaxel, docetaxel, vinblastine, vincristine, vindesine, irinotecan, doxorubicin, epirubicin, leucovorin, etopside, teniposide, idarubicine, gemcitabine, daunorubicin, carboplatin, cisplatin, oxaliplatin, chlorambucil, melphalan, cyclophosphamide, ifosfamide, temozolomide, thiotepa, mitomycin C, busulfan, carmustine, lomustine, 5-fluorouracil, capecitabine, exemestane, methotrexate, trimetrexate, fluorouracil, fluorodeoxyuridine, azacytidine, mercaptopurine, thioguanine, pentostatin, cytarabine, fludarabine, hydroxyurea, bevacizumab, cetuximab, gefitinib and imatinib.

20. The method of claim 17, wherein the cancer is breast cancer, small cell lung carcinoma, colon cancer, non-small cell lung cancer, renal cell cancer, a gastrointestinal stromal tumor, thyroid cancer, a sarcoma or a neuroendocrine tumor.
